(19) United States
(12) Patent Application Publication
(10) Pub. No.: US 2018/0036270 A1
(43) Pub. Date: Feb. 8, 2018

(54) A METHOD OF PROTECTING AGAINST CARBONYL STRESS INDUCED ISCHEMIA-REPERFUSION INJURY IN THE DIABETIC BRAIN VIA ADMINISTRATION OF N-ACETYLCYSTEINE

(71) Applicant: Karen Y STOKES, Shreveport, LA (US)

(72) Inventors: Tak Yee AW, Shreveport, LA (US); Karen Y. STOKES, Shreveport, LA (US)

(73) Assignee: Board of Supervisors of Louisiana State University and Agricultural and Mechanical College, Baton Rouge, LA (US)

(21) Appl. No.: 15/550,149

(22) PCT Filed: Feb. 16, 2016
(86) PCT No.: PCT/US16/18029

§ 371 Date: Aug. 10, 2017

Related U.S. Application Data
(60) Provisional application No. 62/115,855, filed on Feb. 13, 2015.

(51) Int. Cl. A61K 31/198; A61K 45/06; A61K 38/28

(52) U.S. Cl. CPC A61K 31/198; A61K 38/28; A61K 45/06

(57) ABSTRACT

A method for one of preventing and minimizing diabetes pathology in a mammal, comprising administering to the mammal an effective amount of N-acetylcysteine (NAC).

### CROSS REFERENCE TO RELATED APPLICATIONS

This application claims priority to U.S. Provisional Patent Application No. 62/115,855 filed Feb. 13, 2015, the contents of which are incorporated herein by reference in its entirety. To the extent that there is any conflict between the incorporated material and the present disclosure, the present disclosure will control.

### STATEMENT REGARDING FEDERALLY SPONSORED RESEARCH OR DEVELOPMENT

Not applicable.

[symbol]

### FIELD OF THE INVENTION

The present invention relates to the prevention and treatment, via administration of N-acetylcysteine (NAC), of pathophysiologies or diseases that are linked to diabetes mellitus and/or a decreased tissue glutathione (GSH), and specifically relates to

[symbol]

### BACKGROUND

Diabetes mellitus is a clinically important independent risk factor for the development of cardiovascular disease (CVD) and cerebrovascular disease. Globally, diabetes prevalence is almost even among males and females, with prevalence increasing sharply with age. Diabetic patients have 2-4 times greater risk for heart diseases and stroke. Moreover, the clinical outcome in patients with pre-existing CVD or risk is exacerbated by diabetes. CVD patients with diabetes have poorer prognosis and survival than CVD patients without diabetes, and the 5-year mortality rate post myocardial infarction can reach 50% in diabetic individuals. The incidence of stroke has been shown to occur twice as frequently in hypertensive individuals with diabetes than those with hypertension alone and stroke patients with diabetes have a 3-fold higher mortality than their non-diabetic counterparts. During adolescence males tended to be prone to Type 1 and females to type 2 diabetes mellitus. As adults, males exhibit a higher prevalence and severity of diabetic retinopathy, a microvascular complication associated with diabetes. Interestingly however, in coronary heart disease or stroke and stroke-related fatality, diabetic females are more at risk than diabetic males, and importantly this is at all age-groups.

Diabetes-associated heart disease and stroke accounted for over 60% of diabetic fatalities. Equally debilitating for diabetics with long-term poorly controlled blood glucose is a non-age related decline in cognitive function and dementia risk due to a progressive neurological disorder termed diabetic encephalopathy, and subsequent loss of quality of life. Indeed, Alzheimer's disease is reportedly twice as prevalent in diabetics. The plasma glycemic status is typically used as a predictor of increased CVD risk in diabetes, and risk of heart disease and stroke is exaggerated in diabetics with poor glycemic control. However, an increased CVD risk remains even in diabetic individuals with well controlled blood sugar levels, suggesting the involvement of other factors.

### SUMMARY OF THE INVENTION

Wherefore, it is an object of the present invention to overcome the above mentioned shortcomings and drawbacks associated with the current technologies.

Another object of the present invention is to administering NAC as a preventative and protective treatment against brain infarction after ischemia-reperfusion and accelerated onset of thrombosis in brain microvessels of diabetic individuals.

A further object of the present invention is to provide a method for one of preventing and minimizing I/R injury in a mammal, comprising administering to the mammal an effective amount of N-acetylcysteine (NAC), optionally including when the mammal has diabetes, and when the mammal is a human, and when a compound to control glucose is co-administered, and when the effective amount is between one of 40 mg NAC per day per kg mass of the mammal to 80 mg NAC per day per kg mass of the mammal and 80 mg NAC per day per kg mass of the mammal to 160 mg NAC per day per kg mass of the mammal, given individually or combination with lowered doses of current therapies such as insulin or anti-glycemic drugs, and when the mode of administration being orally or intravenously, and when the NAC is administered before disease (prophylactic) in at-risk populations, during active disease/pathology and during disease resolution, and pharmacologically active salts, esters, and derivatives of NAC.

A further object of the present invention is to provide a method for treating decreased tissue GSH in a mammal, comprising administering to the mammal an effective amount of N-acetylcysteine (NAC) or GSH, including when the decreased tissue GSH and/or high glucose utilization, and/or high MG production is associated with a pathophysiology or disease including, but not limited to, cancer, insulin resistance, metabolic diseases (obesity etc.), thrombotic/thromboembolic pathologies, neurodegenerative disorders (dementia, Alzheimer's and Parkinson's), and planned surgical ischemic episodes (e.g. transplant, bypass).

A further object of the present invention is to provide a method for one of preventing and minimizing diabetes pathology in a mammal, comprising administering to the mammal an effective amount of N-acetylcysteine (NAC), including when the diabetes pathology is one of associated microvascular disorders, including nephropathy, retinopathy and/or neuropathy, associated macrovascular disorders, including peripheral artery disease, CVD, and associated glycemic control disorders due to diabetes-associated glucose memory and epigenetic changes.

A further object of the present invention is to provide a method for one of preventing and minimizing conditions that are associated with elevated MG in a mammal, comprising, administering to the mammal an effective amount of N-acetylcysteine (NAC), including when the condition includes: cataracts, uremia, peritoneal dialysis and liver cirrhosis.

A further object of the present invention is treat diabetes comprising administering NAC and a further compound to control glucose levels, including when the further compound is insulin and when the compound is one of biguanides, such as, metformin, metformin liquid, and metformin extended release, sulfonylureas, such as glimepiride, glyburide, glipizide, and micronized glyburide, meglitinides, such as repaglinide, D-phenylalanine derivatives, such as nateglinide, thiazolidinediones, such as pioglitazone, DPP-4 inhibitors, such as sitagliptin, saxagliptin, and linagliptin, alpha-glucosidase inhibitors, such as acarbose and miglitol, bile acid sequestrants, such as colesevelam, and combination pills/administrations, such as pioglitazone and metformin, glyburide and metformin, glipizide and metformin, sitagliptin and metformin, saxagliptin and metformin, repaglinide and metformin, and pioglitazone and glimepiride.

A further object of the present invention is to protect proteins from glycation.

A further object of the present invention is to treat and decrease leakage across the blood brain barrier.

A further object of the present invention is to protect vascular integrity against damage caused by diabetes.

A further object of the present invention is to protect against and minimize diabetes related arterial and venule thrombosis.

A further object of the present invention is to protect and treat diabetes related accelerated coagulation.

A further object of the present invention is normalization of exacerbated platelet-leukocyte aggregate formation.

Diabetes is characterized by endothelial dysfunction, hyperglycemia and elevated plasma levels of methylglyoxal (MG), a glucose-derived potent glycating agent. Diabetes increases reactive oxygen species (ROS) levels, compromises the antioxidant defense enzymes, and attenuates the levels of intracellular antioxidants. This creates an environment of increased oxidative stress and carbonyl stress.

Methylglyoxal (MG) is a highly reactive dicarbonyl metabolite of α-oxoaldehydes which are potent glycating agents. Carbonyl stress results from enhanced MG generation and accumulation of MG-glycated proteins. MG is known to mediate neurodegenerative CNS pathology. MG-mediated glycation of extracellular matrix laminin and fibronectin and the accumulation of glycated products in the endoneurium inhibited neurite outgrowth from sensory neurons in streptozotocin (STZ)-induced diabetic rat brain. In addition, the glycation reaction of MG with amino acids can generate superoxide radical anion. As a result, protein damage by MG can be mediated by carbonyl stress through formation of protein carbonyls, as well as by oxidative stress through enhanced ROS formation.

Moreover, treatment with 100 μM MG in rat thoracic aortic rings for 24 h decreases acetylcholine-induced vascular relaxation via a reduction in p-eNOS (Ser1177) and p-AMPKα (Thr172). MG can reduce endothelial angiogenesis through RAGE-mediated, ONOO(⁻)-dependent and autophagy-induced VEGFR2 degradation. The inventors found that MG can increase human brain microvascular endothelial cell barrier permeability as measured by loss of transendothelial electrical resistance. This is exacerbated by GSH synthesis inhibition and prevented by NAC. The enhanced MG concentration in diabetic patients can also directly contribute to the platelet dysfunction associated with diabetes. Platelet dysfunction is characterized by hyperaggregability and reduced thrombus stability via potentiating thrombin-induced platelet aggregation and dense granule release, but inhibiting platelet spreading on fibronectin and collagen. Given that endothelial dysfunction and enhanced platelet activation could promote stroke, these studies indicate that MG can contribute to ischemic brain injury.

GSH, the co-factor in the elimination of MG, is significantly decreased after brain ischemia/reperfusion (I/R) injury. The importance of this is supported by the fact that brain infarction and edema were exacerbated further in the presence of L-buthionine sulfoximine (BSO), a selective inhibitor of glutamate cysteine ligase (GCL), the rate limiting enzyme in GSH production. This suggests that the endogenous brain GSH content is an important determinant in the defense mechanisms against lesion formation after ischemia. The levels of GSH also significantly decrease in insulin-dependent diabetes, which maybe the reason that diabetes potentiates post-ischemic brain injury. GCL is comprised of the catalytic subunit (GCLc) and modulatory subunit (GCLm). The expression of GCLc and GCLm is dramatically reduced in mesenteric vessels of db/db type II diabetic mice compared with db/m non-diabetic mice. Additionally, GCLc is significantly decreased in the retina of STZ-induced diabetic rats. But very little is known about changes in GCL in diabetic brain and after I/R.

MG elimination is catalyzed by the glyoxalase system that comprises the glyoxalase I and II (Glo I and II) enzymes. But the current studies by other researchers about the expression and activity of these enzymes in diabetes are contradictory, i.e., there are evidence that support an increase in Glo I and Glo II, a decrease in Glo I, or no change in either enzymes. Therefore, the inventors endeavored to further clarify the expression and function of the glyoxalase system in diabetes.

The inventors sought, among other things, to determine if diabetes enhances I/R brain injury, the contribution of MG and GSH, the mechanism of GSH decrease including the expression of GCLc and supply of cysteine substrate, and the changes in the expression and activity of the glyoxalase enzymes. Because of the relatively few studies of stroke in diabetic mouse models, and the initial use of type I DM to test the concept, the inventors developed two models of diabetes-induced exacerbation of stroke, one chemical and one genetic.

### BRIEF DESCRIPTION OF THE DRAWINGS

The accompanying drawings, which are incorporated in and constitute a part of the specification, illustrate various embodiments of the invention. The general description of the invention given above and the detailed description of the drawings given below, serve to explain the principles of the invention. It is to be appreciated that the accompanying drawings are not necessarily to scale since the emphasis is placed on illustrating the principles of the invention. The invention will now be described, by way of example, with reference to the accompanying drawings which:

FIGS. 1A-1C are a two graphs (FIGS. 1A and 1C) and six photos (FIG. 1B) demonstrating ischemia/reperfusion injury in control and diabetic mouse brains;

FIGS. 2A and 2B are a bar graph of plasma glucose levels of vehicle-treated and STZ-treated mice at 4 weeks post STZ (FIG. 2A) and line graph of percent brain infarct area in vehicle- and STZ treated mice to plasma glucose level;

FIGS. 3A-3D is are bar graphs of GSH and MG concentrations in vehicle controls and STZ-treated diabetic mice (FIGS. 3A and 3C) and line graphs of percent brain infarct area to tissue GSH levels (FIG. 3B) and to the ratio of tissue MB level to tissue GSH level;

FIGS. 4A and 4B are two bar graphs showing percent infarct area (FIG. 4A) and brain GSH levels (FIG. 4B) of GCLm^{+/+} and GCLm^{-/-} mice

FIGS. 5A-5E are two bar graphs showing percent infarct area for STZ treated diabetic mice and STZ treated diabetic mice given NAC for 1 week (FIG. 5B) and STZ treated diabetic mice and STZ treated diabetic mice given NAC for 3 weeks (FIG. 5A), two bar graphs showing brain GSH concentrations for STZ treated diabetic mice and STZ treated diabetic mice given NAC for 1 week (FIG. 5D) and STZ treated diabetic mice and STZ treated diabetic mice given NAC for 3 weeks (FIG. 5C), and a line graph of percent brain infarct area to tissue GSH levels;

FIGS. 6A-C are three bar graphs showing brain GCL activity in vehicle and STZ treated mice (FIG. 6A), brain cysteine concentrations (nmol/mg protein) for vehicle mice, STZ treated mice, and STZ treated mice given NAC for three weeks (FIG. 6B), and brain cysteine concentrations for vehicle mice given NAC and vehicle mice given BSO (FIG. 6C);

FIGS. 7A-7C are two bar graphs showing plasma glucose levels (FIG. 7A) and brain MG levels (FIG. 7B) for vehicle mice, STZ treated mice, and STZ treated mice given NAC for three weeks, and one bar graph showing MG-to-GSH ratio for STZ treated mice and STZ treated mice given NAC for three weeks (FIG. 7C);

FIGS. 8A-8D are a set of Western blots of the expression of occludin, GCLc, MG, and actin for diabetic and vehicle mice (FIG. 8A), and three bar graphs of occludin expression (FIG. 8B), GCLc expression (FIG. 8C), and anti-MG expression (FIG. 8D) for vehicle mice and STZ treated mice;

FIGS. 9A and 9B are two bar graphs showing Glo I activity (FIG. 9A) and Glo II activity (FIG. 9B) for vehicle mice and STZ treated mice;

FIG. 10 is a bar graph showing protein carbonyl contents of vehicle mice, and STZ treated mice two and four weeks after onset of diabetes, and STZ treated mice two weeks after onset of diabetes given NAC for one week;

FIG. 11 is a diagram of the structure of NAC;

FIGS. 12A-12C are two bar graphs (FIGS. 12A and 12C) and three photos (FIG. 12B) demonstrating ischemia/reperfusion injury in control and diabetic mouse brains;

FIGS. 13A and 13B are two bar graph showing time to onset of thrombosis in arterioles (FIG. 13A) and venules (FIG. 13B) for vehicle mice, 20 week diabetic (STZ treated) mice and 20 week diabetic (STZ treated) mice given NAC for one week;

FIGS. 14A-14C are two bar graphs showing brain GSH levels (FIG. 14A) and MG levels (FIG. 14B) for vehicle mice, STZ treated mice, and STZ treated mice given NAC for three weeks, and a bar graph of brain protein carbonyl levels of for vehicle mice, STZ treated mice two and four weeks after onset of diabetes, and STZ treated mice given NAC for one week (FIG. 14C);

FIG. 15 is a line graph percent infarct area for different MG to MGH ratios;

FIGS. 16A-16E are four photos showing immunohistochemical staining of brain microvessels occludin expression for control (FIG. 6A) and diabetic (FIG. 6C) and glycated protein adducts for control (FIG. 6B) and diabetic (FIG. 6D), and a bar graph showing percent stained vessels for occludin and MG for control and diabetic (FIG. 16E);

FIGS. 17A-17D are six photos showing immunohistochemical staining of brain microvessels for E-selectin (FIG. 17A), ICAM-1 (FIG. 17B), and VCAM-1 (FIG. 17C) for a diabetic and a control mouse, and one bar graph showing percent stained vessels for E-selectin, ICAM-1, and VCAM-1 for the diabetic the control mouse (FIG. 17D);

FIG. 18 is a Western blot of MG glycated protein adduct for human brain microvascular endothelial cells grown in normal glucose, high glucose for seven or twelve days, and acutely fluctuating glucose;

FIG. 19 is a Western blot of MG glycated protein adduct for human brain microvascular endothelial cells grown in control conditions and with a four hour treatment of MG with and without 2 mM of NAC;

FIGS. 20A-20F are three bar graphs showing plasma glucose (FIG. 20A) brain GSH (FIG. 20C) and brain MG (FIG. 20D) levels in diabetic and control mice, and three line graphs of percent brain infarct area to plasma glucose (FIG. 20B), brain GSH level (FIG. 20E), brain MG (FIG. 20F) levels;

FIGS. 21A-21E are four Western blots paired to bar graphs for MG adducts (FIG. 21A), occludin (FIG. 21B), GCLc (FIG. 21C), and occludin-MG (FIG. 21D) for vehicle mice, STZ treated mice, and STZ treated mice given NAC for three weeks, and one bar graph of blood brain barrier permeability for vehicle mice, STZ treated mice, and STZ treated mice given NAC for three weeks;

FIGS. 22A-22D are two bar graphs showing time to onset of thrombosis in venules (FIG. 22A) and arterioles (FIG. 22C) for vehicle mice, six week diabetic (STZ treated) mice, and six week diabetic (STZ treated) mice given NAC for three weeks, and two bar graphs showing time to cessation of thrombosis in venules (FIG. 22B) and arterioles (FIG. 22D) for vehicle mice, six week diabetic (STZ treated) mice, and six week diabetic (STZ treated) mice given NAC for three weeks;

FIGS. 23A-23D are two bar graphs showing time to onset of thrombosis in venules (FIG. 23A) and arterioles (FIG. 23C) for vehicle mice, twenty week diabetic (STZ treated) mice, and twenty week diabetic (STZ treated) mice given NAC for three weeks, and two bar graphs showing time to cessation of thrombosis in venules (FIG. 23B) and arterioles (FIG. 23D) for vehicle mice, twenty week diabetic (STZ treated) mice, and twenty week diabetic (STZ treated) mice given NAC for three weeks;

FIG. 24 is a bar graph showing tail bleed time for vehicle mice, twenty week diabetic (STZ treated) mice, and twenty week diabetic (STZ treated) mice given NAC for three weeks;

FIGS. 25A-25D are bar graphs showing percent of aggregate formation between platelets and circulating leukocytes (FIG. 25A), lymphocytes (FIG. 25B), neutrophils (FIG. 25C) and monocytes (FIG. 25D) for vehicle mice, five week diabetic (STZ treated) mice, and five week diabetic (STZ treated) mice given NAC for two weeks;

FIGS. 26A-26D are bar graphs showing percent of aggregate formation between platelets and circulating leukocytes (FIG. 26A), lymphocytes (FIG. 26B), neutrophils (FIG. 26C) and monocytes (FIG. 26D) for vehicle mice, nineteen week diabetic (STZ treated) mice, and nineteen week diabetic (STZ treated) mice given NAC for two weeks;

FIG. 27 is a set of four bar graphs for number of platelets/ml for vehicle mice and four week diabetic (STZ treated) mice for total, immature, mature, and TO⁺, JON/A⁺;

FIGS. 28A-28C are three bar graphs showing percent platelet-neutrophil aggregate formation (FIG. 28A), time to onset of thrombosis (FIG. 28B), and time to cessation of thrombosis (FIG. 28C) for Akita non-diabetic mouse, Akita diabetic mouse, Akita diabetic mouse plus insulin, and Akita diabetic mouse plus insulin and NAC; and

FIG. 29 is a bar graph of time to cessation of thrombosis for nondiabetic mice with and without transient ischemic attacks, diabetic mice with and without transient ischemic attacks, and diabetic mice given NAC with ischemic attacks.

### DETAILED DESCRIPTION OF THE INVENTION

The present invention will be understood by reference to the following detailed description, which should be read in conjunction with the appended drawings. It is to be appreciated that the following detailed description of various embodiments is by way of example only and is not meant to limit, in any way, the scope of the present invention.

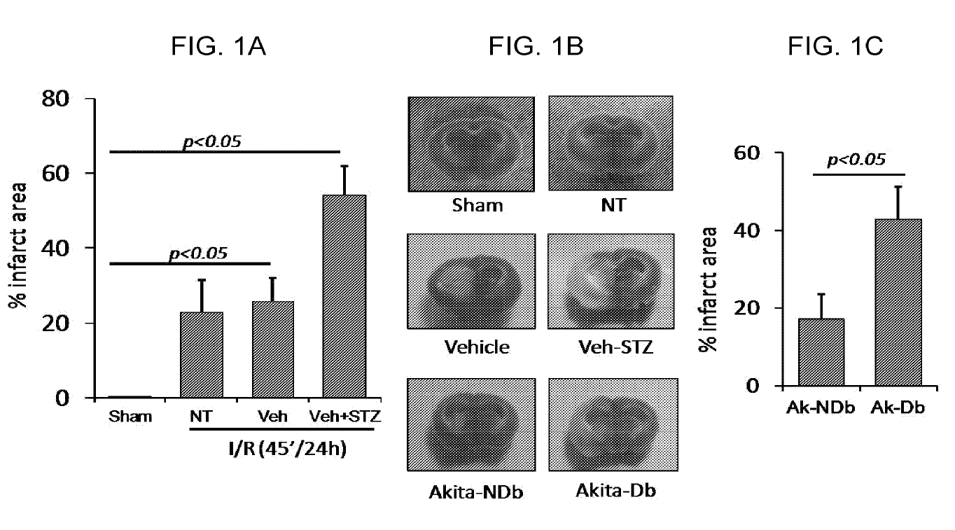

Turning now to FIGS. 1A-1C, a brief description concerning the various components of the present invention will now be briefly discussed. As shown, ischemia/reperfusion (I/R) injury in control and diabetic brain was determined using the middle cerebral artery occlusion-reperfusion (MCAoR) model of Koizumi. MCA occlusion was for 45 min and reperfusion for 24 h. Diabetes model were: mice injected with streptozotocin (STZ) or Akita mice lacking the Insulin-2 gene which spontaneously developed diabetes in 4 wks. Results are mean±SE. NT=no treatment; Veh=citrate buffer; Veh+STZ=4 week post STZ. Ak—Akita. The post I/R infarct area is represented by the unstained (white) regions of left brain sections.

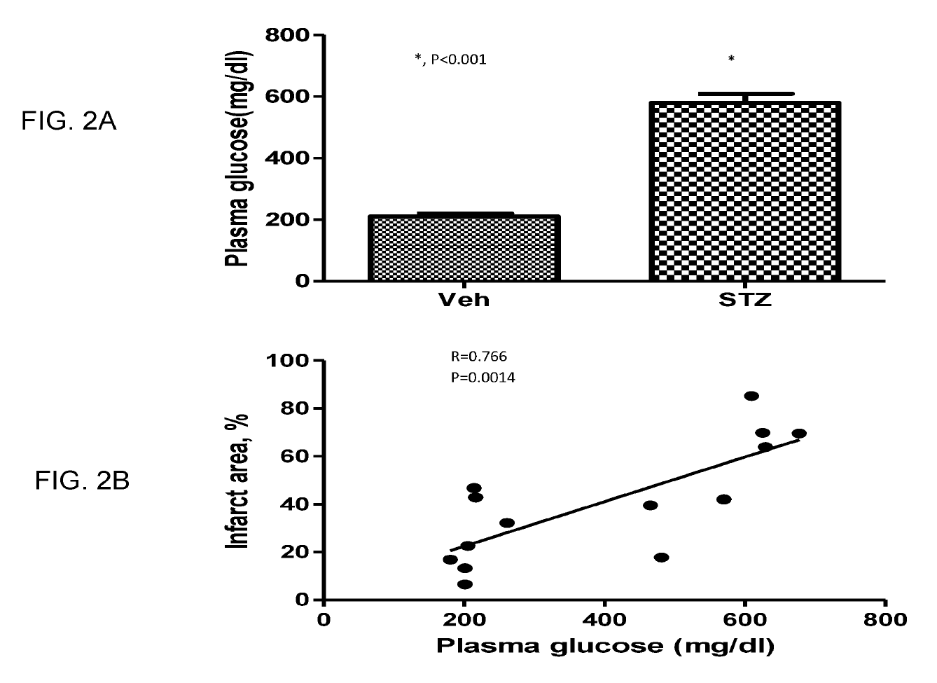

As shown in FIGS. 2A and 2B, plasma glucose levels were determined in vehicle-treated and STZ-treated mice at 4 weeks post STZ. Plasma levels in diabetic mice were elevated at ˜600 mg/L which was 3-fold higher than controls (˜200 mg/dL). The percent (%) brain infarct area in vehicle- and STZ treated mice is linearly correlated with the plasma glucose level.

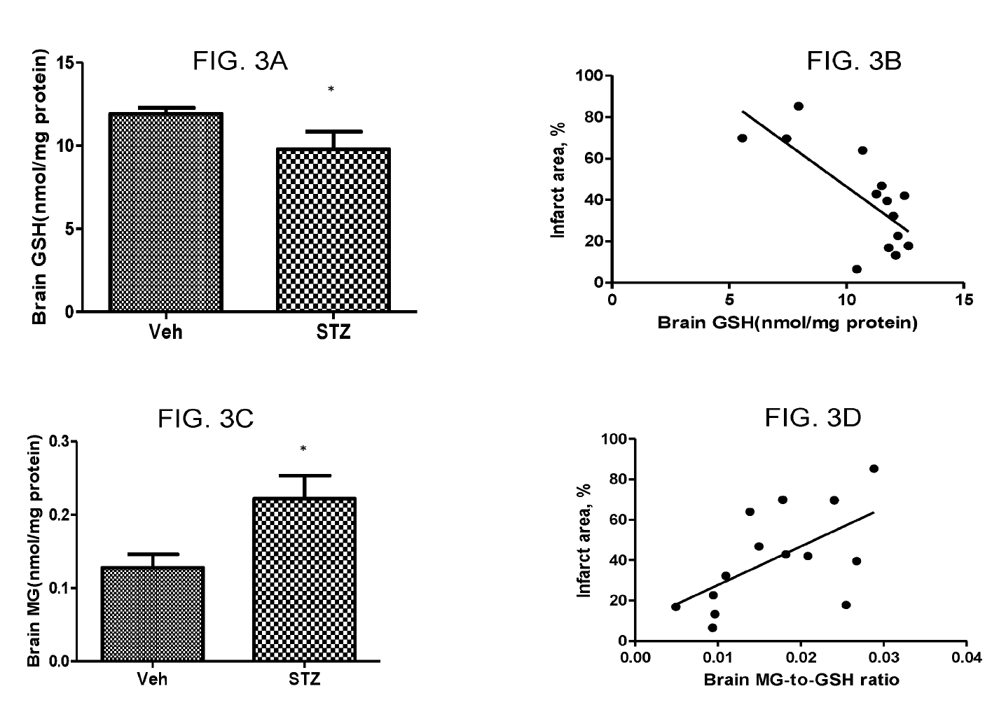

As shown in FIGS. 3A-3D, brain GSH and MG concentrations in vehicle controls and STZ-treated diabetic mice were determined by HPLC. Tissue GSH levels in diabetic brains were significantly lower as compared to vehicle-treated controls. The percent (%) brain infarct area was negatively correlated with tissue GSH contents. Tissue MG levels were significantly elevated in diabetic mouse brain as compared to vehicle controls. Interestingly, percent infarct area was not correlated with tissue MG levels per se (not shown). However, the percent brain injury corresponded to the MG-to-GSH ratio, suggesting that I/R-induced brain injury in the diabetic brain was a function of the GSH potential for MG elimination.

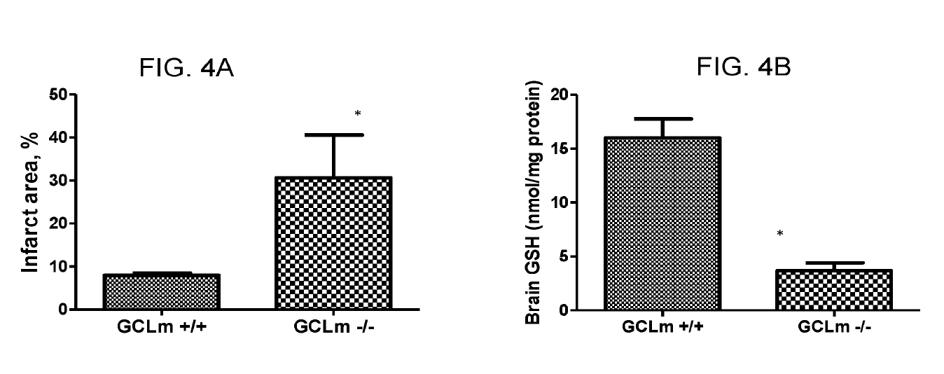

Turning to FIGS. 4A and 4B, GCLm^{+/+} mice showed significant increasing GSH levels and decreasing infarct area compared with GCLm^{-/31} mice.

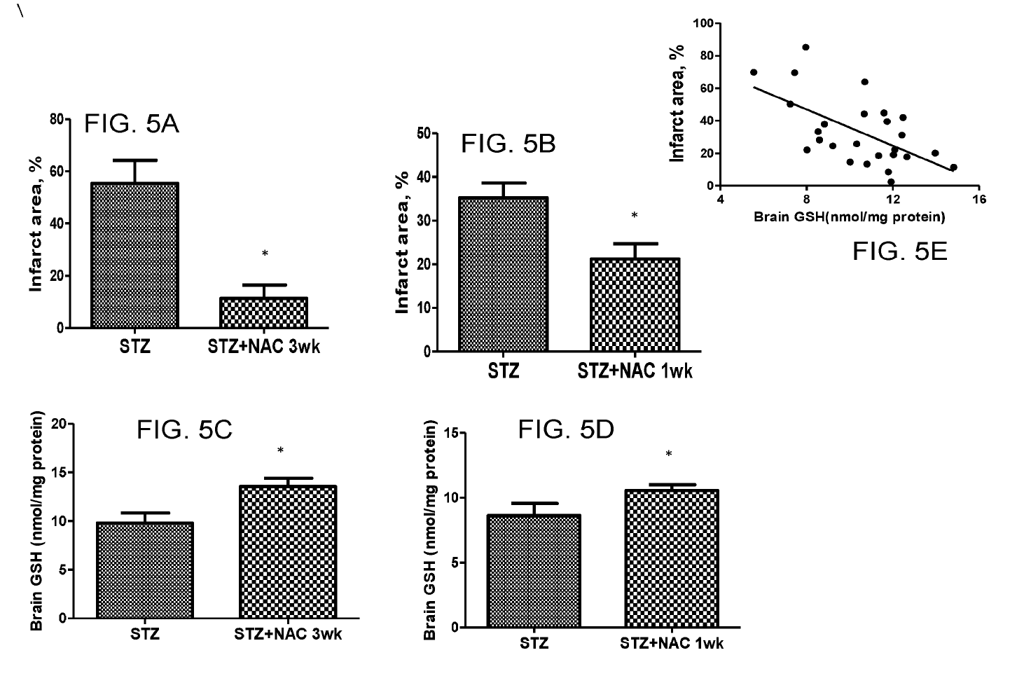

Turning to FIGS. 5A-5E, vehicle- and STZ-treated diabetic mice were given 2 mM N-acetylcysteine (NAC) in the drinking water for 1 or 3 weeks before MCAoR surgery and induction of brain I/R injury. Results show that infarct area was significantly attenuated in NAC-treated diabetic mice. Brain GSH concentrations were significantly higher in the NAC-treated group. A significant negative correlation was obtained between percent infarct area and GSH levels.

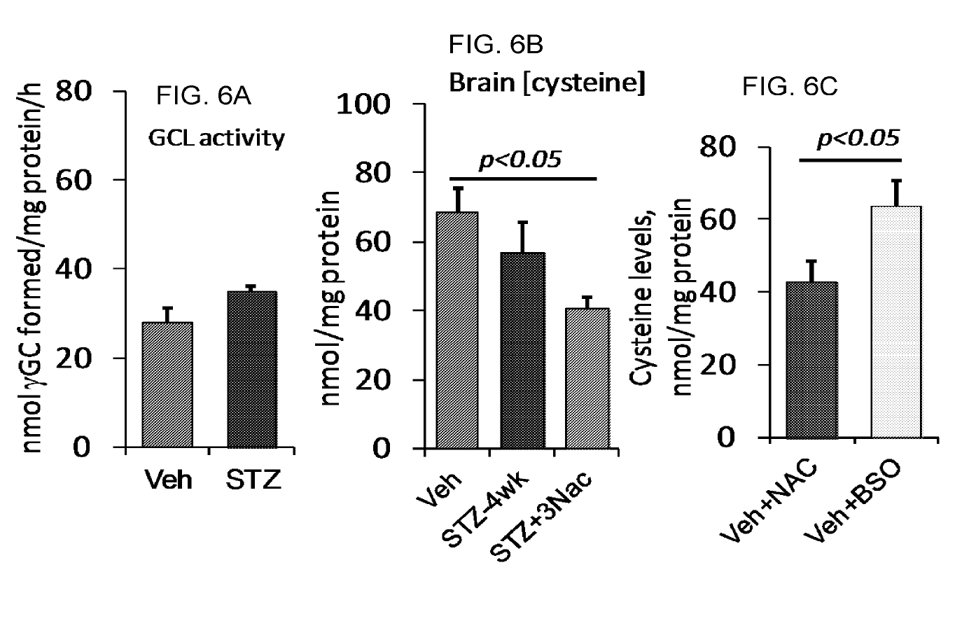

Turning to FIGS. 6A-6C, FIG. 6A shows brain GCL activity was measured by the formation of γ-glutamylcysteine and tissue cysteine levels quantified by HPLC. The results show no difference in GCL activity between vehicle- or STZ-treated mice at 4 weeks, suggesting that diabetes did not alter GCL protein content. GCLc protein expression was examined by Western blot analysis. FIG. 6B shows cysteine concentrations in diabetic brain tended to be lower than vehicle control (although not significant). However, brain cysteine levels were significantly decreased in NAC-treated animals, suggesting that cysteine was being utilized for enhanced GSH synthesis (see FIG. 5), and that a significant source of cysteine came from NAC. FIG. 6C shows inhibition of GSH synthesis by BSO blocked the NAC effect and resulted in higher cysteine levels. This latter result confirms that NAC was an important cysteine source for GSH synthesis.

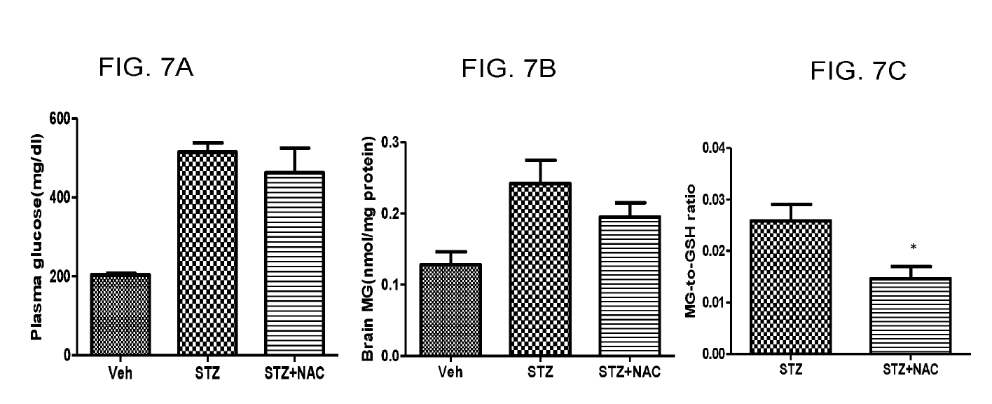

Turning to FIGS. 7A- 7C, Treatment of mice with 2 mM NAC for 3 weeks did not alter plasma glucose levels in 4 week diabetic animals, shown in FIG. 7A, suggesting that NAC had no effect on plasma glucose. Shown in FIG. 7B, in NAC-treated mice, brain MG levels remained high, although trending lower. The finding of elevated MG largely reflected the high plasma glucose, whose continued metabolism yields MG. Shown in FIG. 7C, when expressed as MG-to-GSH ratio (GSH data from FIG. 5), it is noted that NAC significantly decreased this ratio, suggesting that NAC enhanced the potential for MG elimination by increasing tissue GSH content.

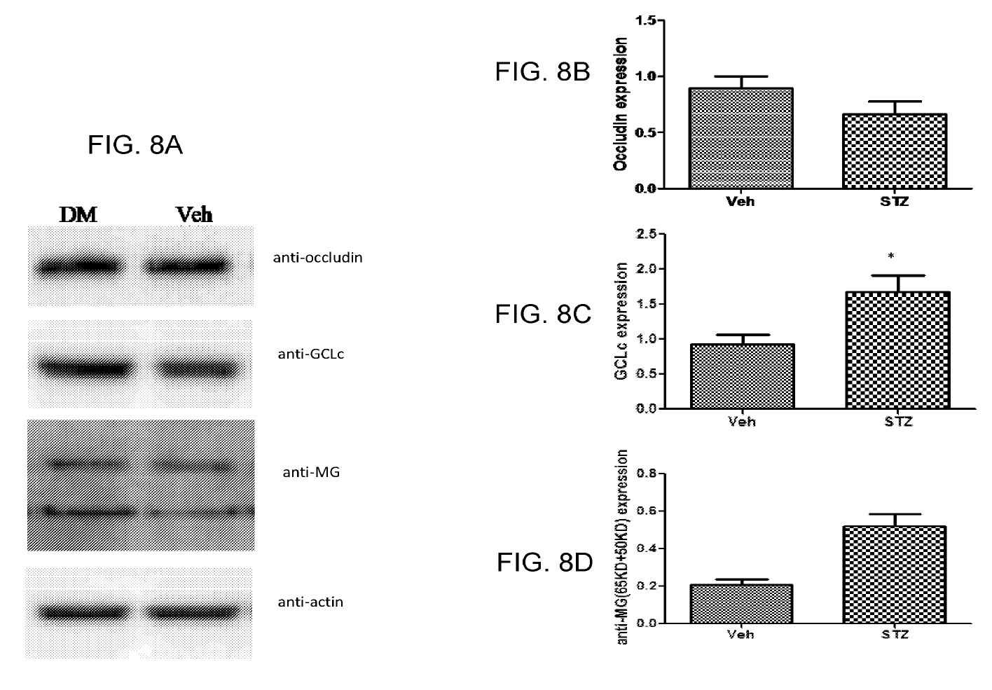

Turning to FIGS. 8A-8D, the expression of occludin, GCLc and protein-MG by western blot is shown. The occludin expression had a decreasing tendency in diabetic mice compared with Veh mice. The GCLc expression was significantly increased in diabetic mice compared with Veh mice (P<0.05). The two primary bands whose molecular size were same as occludin and GCLc were significantly increased in diabetic mice compared with Veh mice (P<0.05), which indicated protein-glycation was significantly enhanced in diabetes.

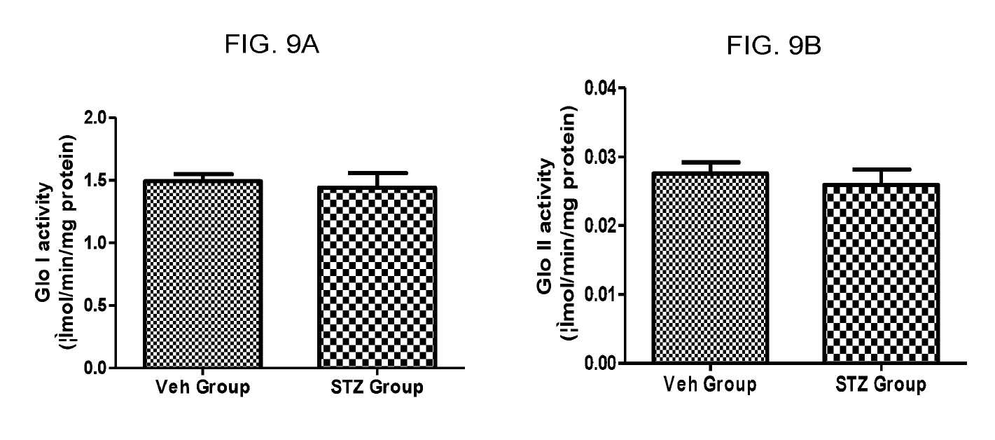

Turning to FIGS. 9A-9B, Glo I and II activity was measured by SDL formation and GSH regeneration. The results show that diabetes did not change the activity of Glo I & II compared with Veh control mice.

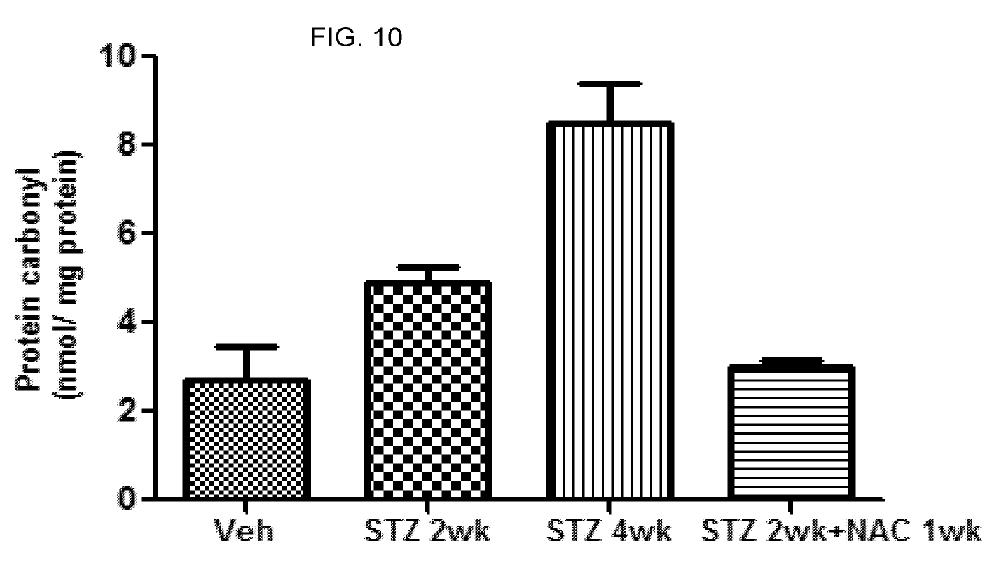

Turning to FIG. 10, the protein carbonyl contents were determined by colorimetric method. Diabetes significantly enhanced protein carbonyl contents regardless of 2 week or 4 week after diabetes onset compared with control mice (both P<0.05). The levels was further increased at 4 week diabetic duration compared with that in 2 week. NAC decreased significantly the levels of protein carbonyl only treating for 1 week.

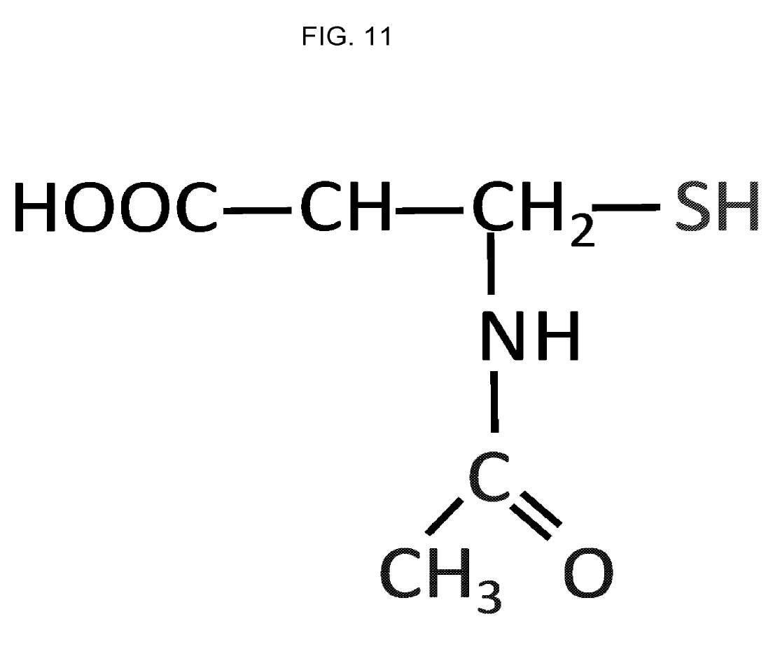

Turning to FIG. 11, the structure of N-acetylcysteine (NAC) is shown. NAC has a chemical formula of C₅H₉NO₃S. The basic backbone is the cysteine amino acid with an N acetyl group—the CH₃CO group shown at the bottom of the structure. The N acetyl group presence renders the molecule less oxidizable than cysteine. The redox active group is the SH (sulfhydryl or thiol moiety) shown on the right side of the structure. Acting outside of cells, NAC can split disulfide bonds such as in mucus. Within cells, NAC is metabolized to cysteine that enhances GSH synthesis. Only L-NAC is believed to be active.

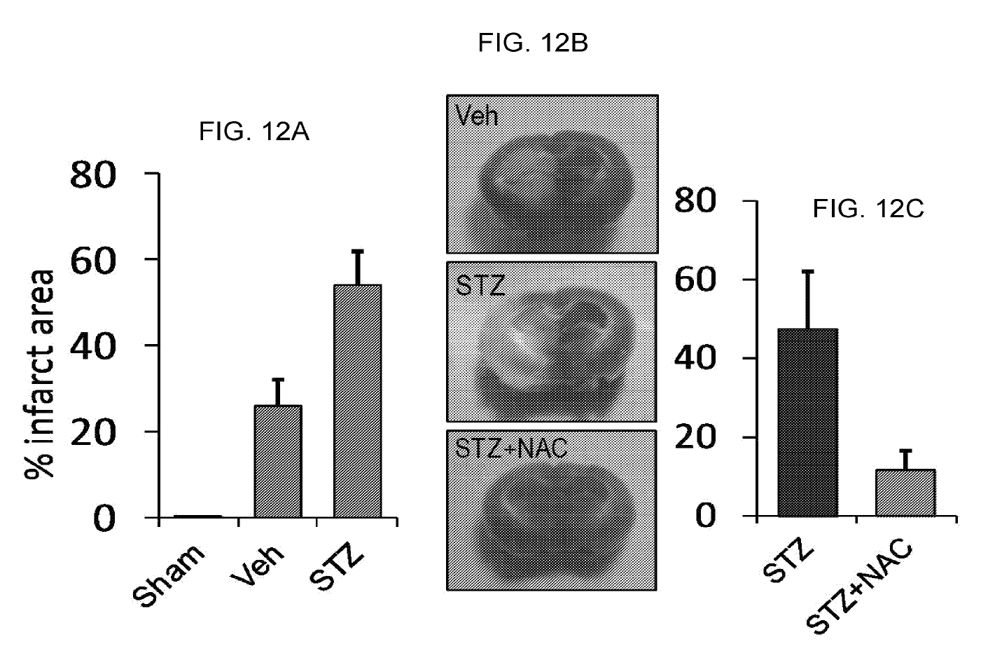

Turning to FIGS. 12A-12C, I/R injury in control and diabetic brain was determined using the middle cerebral artery occlusion-reperfusion (MCAoR) model of Koizumi. MCA occlusion was for 45 min and reperfusion for 24 h. Diabetes was induced with streptozotocin (STZ) and stroke was induced in 4-week diabetic mice. NAC (2 mM) was given in drinking water for 3 weeks. Results are mean±SE. Veh=citrate buffer; STZ=4 week post STZ. The post I/R infarct area is represented by the unstained (white) regions of left brain sections after staining with TTC.

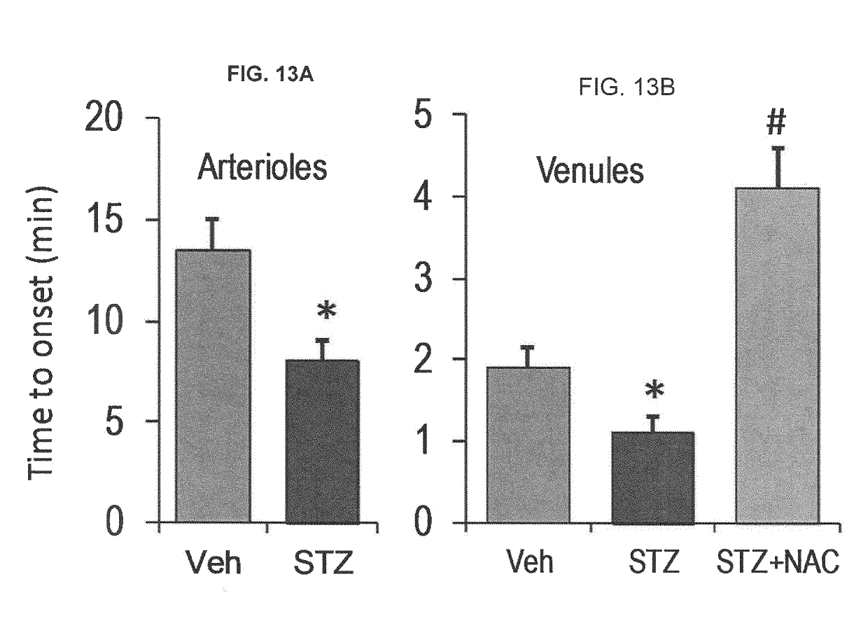

As shown in FIGS. 13A and 13B, cerebral microvessels are more vulnerable to thrombosis in advanced diabetes. Using light-dye induced thrombosis, cerebral arterioles (FIG. 13A) and venules (FIG. 13B) in 20 week diabetic mice (STZ) exhibited accelerated onset of thrombus formation vs. vehicle controls. NAC (2 mM) was given in drinking water for 1 week*P<0.05 vs vehicle controls. For venules, # P<0.05 vs STZ

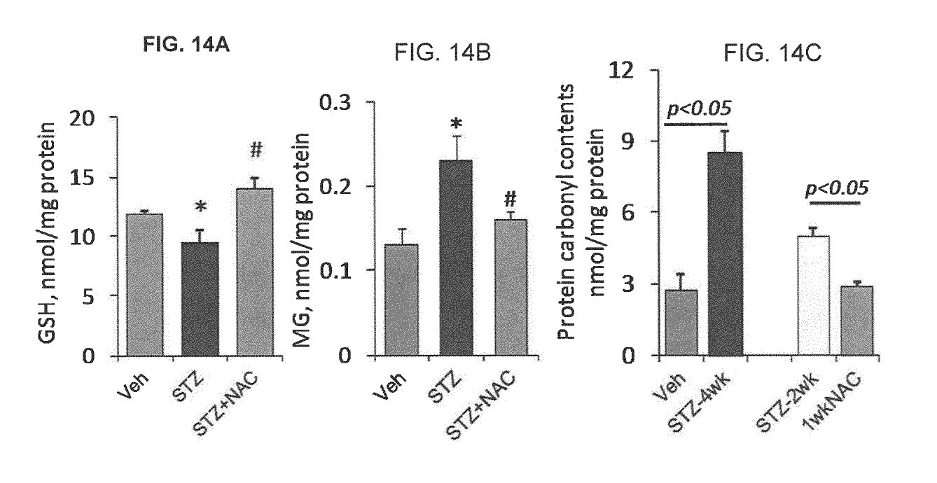

Turning next to FIGS. 14A-14C, brain GSH concentrations in vehicle controls and STZ-treated diabetic mice (4-week diabetes) were determined by HPLC. Tissue GSH levels in diabetic brains were significantly lower than vehicle controls, and GSH levels were increased by NAC treatment for 3 weeks (FIG. 14A). Shown in FIG. 14B, tissue MG levels were determined by HPLC and diabetic brain exhibited significantly higher MG levels that were attenuated by 3 week NAC treatment. Shown in FIG. 14C, brain contents of protein carbonyls, as determined spectrophotometrically, were 3-fold higher than in control brain. Notably, brain protein carbonyls were elevated as early as 2 weeks diabetes, and remarkably, was significantly attenuated after only 1 week NAC treatment.

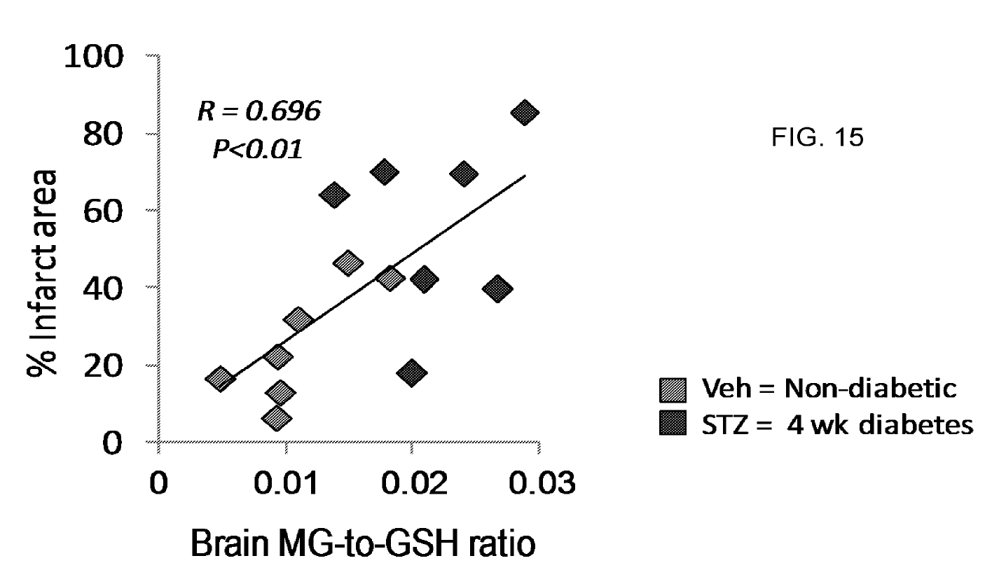

As shown in FIG. 15, although MG levels were significantly elevated in diabetic mouse brain (see FIG. 14B), post I/R brain injury, expressed as percent infarct area was, interestingly not well correlated with tissue MG levels per se (not shown). Rather, percent brain infarct area directly corresponded to the MG-to-GSH ratio, suggesting that I/R-induced brain injury in the diabetic brain was a function of the GSH potential for MG elimination. This means that net formation of protein carbonyls is largely due to the availability of GSH in handling free MG. Therefore, ability of NAC to increase GSH production underscores its effectiveness in attenuating carbonyl stress during diabetes.

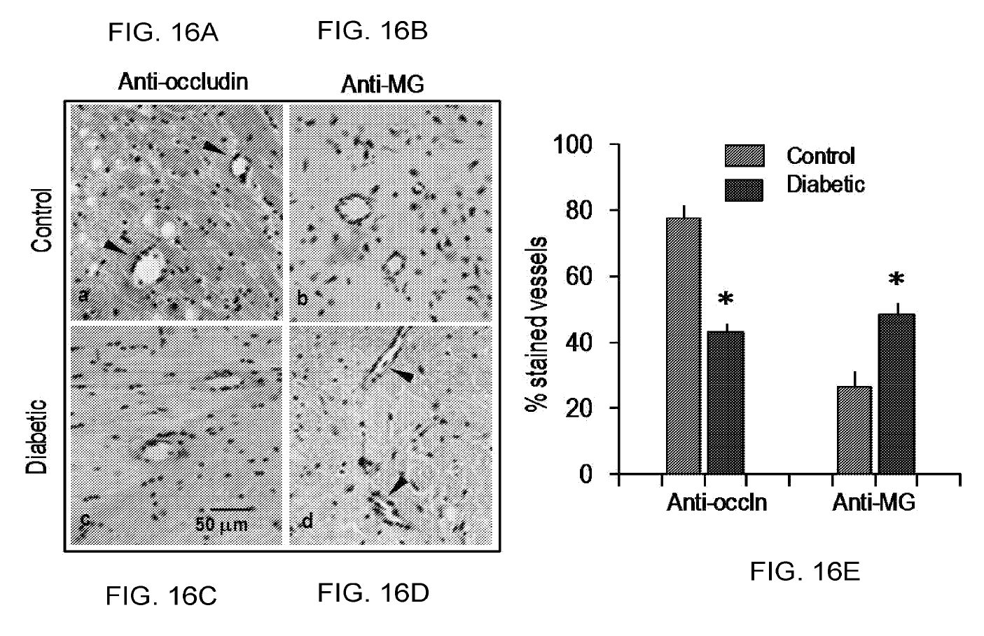

Turning to FIGS. 16A-16E, immunohistochemical staining revealed that diabetic brain microvessels are associated with decreased occludin expression (FIG. 16C) but increased glycated protein adducts (FIG. 16D) as compared to controls (FIGS. 16A and 16B, respectively.) Occludin- or MG-positive cells in representative cerebral microvessels exhibit brown staining (arrowheads) in original photos. The number of positive microvessels is expressed as a percent of total vessels counted (˜50-60). Cell nuclei are stained blue in the original photos with DAPI.

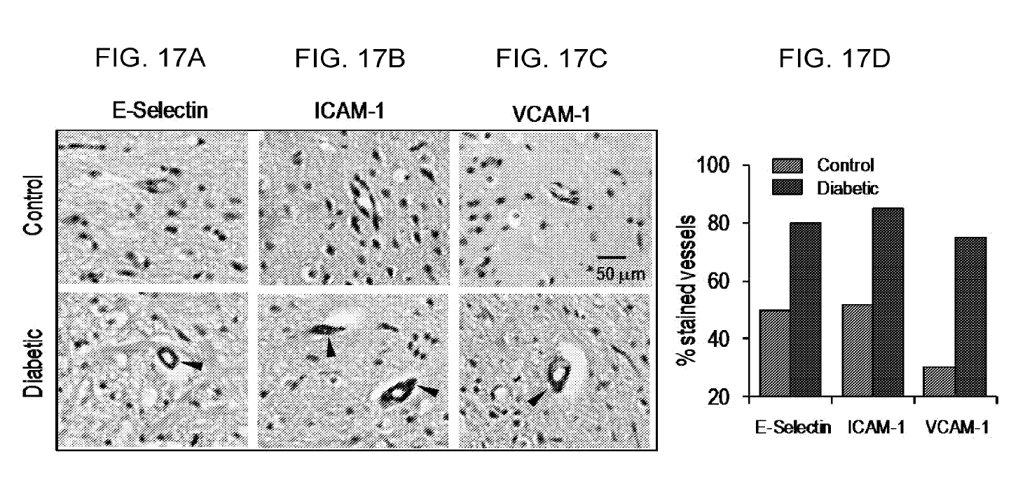

Turning to FIGS. 17A-17D, the diabetic brain exhibits increased expression of endothelial cell adhesion molecules (ECAMs), namely, E-selectin, ICAM-1, and VCAM-1. Expression of ECAMs in microvessels in control and diabetic mouse brain was examined by immunohistochemistry. Arrowheads indicate positive (brown) stain for each of the 3 ECAMs in representative cerebral microvessels. The number of stain-positive microvessels is expressed as a percent of total vessels counted (36-50, 47-54, and 12-40, respectively for E-selectin, ICAM-1 and VCAM-1 from one control and one diabetic mouse). Cell nuclei are stained blue with DAPI.

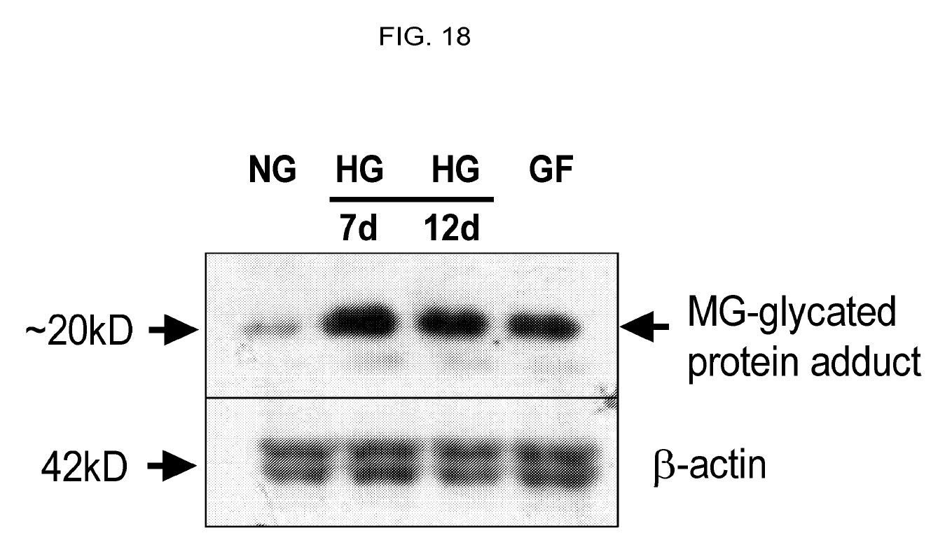

Turning to FIG. 18, human brain microvascular endothelial cells (IHECs) were grown in high glucose (HG) for 7 or 12 days or exposed to acute changes in glucose status (glucose fluctuation, GF). Cells subjected to GF were grown in 25 mM glucose for 7 days and then transferred to normal glucose (NG, 5 mM) for 4 h. Cell extracts were prepared and western blot analysis using anti-MG antibody revealed that HG and GF conditions induced marked increases in MG-adduct formation in a protein band of molecular size ˜20 kD that corresponded to that of histone 3. This suggests that histone 3 is a susceptible target of glycation with important implications for post-translational regulation of NFkB-dependent inflammatory gene expression. In a broader perspective, this finding supports a potentially new paradigm that MG-glycation of histone 3 is a significant post-translational modification in the epigenetic control of gene activation and silencing in various pathologies or disease states.

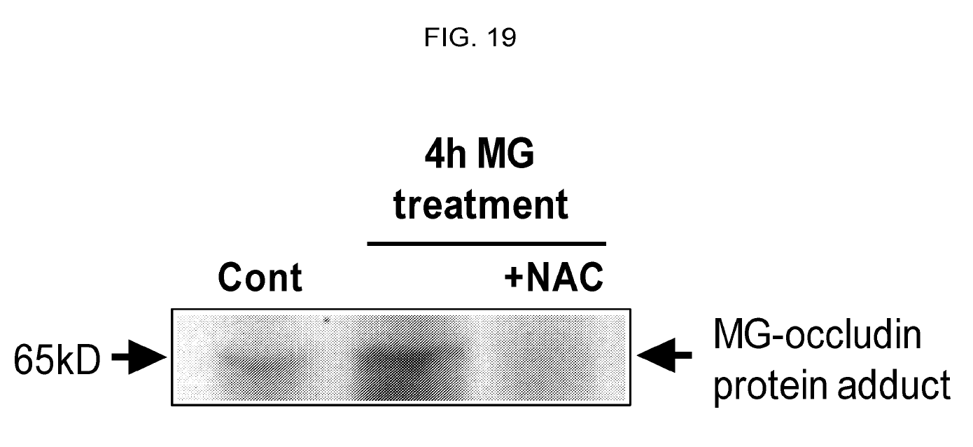

Turning to FIG. 19, IHECs were grown in normal glucose (5 mM) and exposed to MG for 4 h in the absence or presence of 2 mM NAC. Cell extracts were prepared, and Western blot analysis using anti-MG antibody revealed that MG induced adduct formation in a protein of molecular size 65 kD that corresponded to that of occludin. The MG-glycated-occludin adduct was confirmed by immunoprecipitation occludin with anti-occludin antibody followed Western blot analysis with anti-MG. Significantly, occludin glycation was prevented by NAC. This suggests that occludin is a target of MG glycation, and that NAC, likely through GSH-dependent elimination of MG, can effectively abrogate the formation of occludin-adduct with MG, and attenuate carbonyl stress.

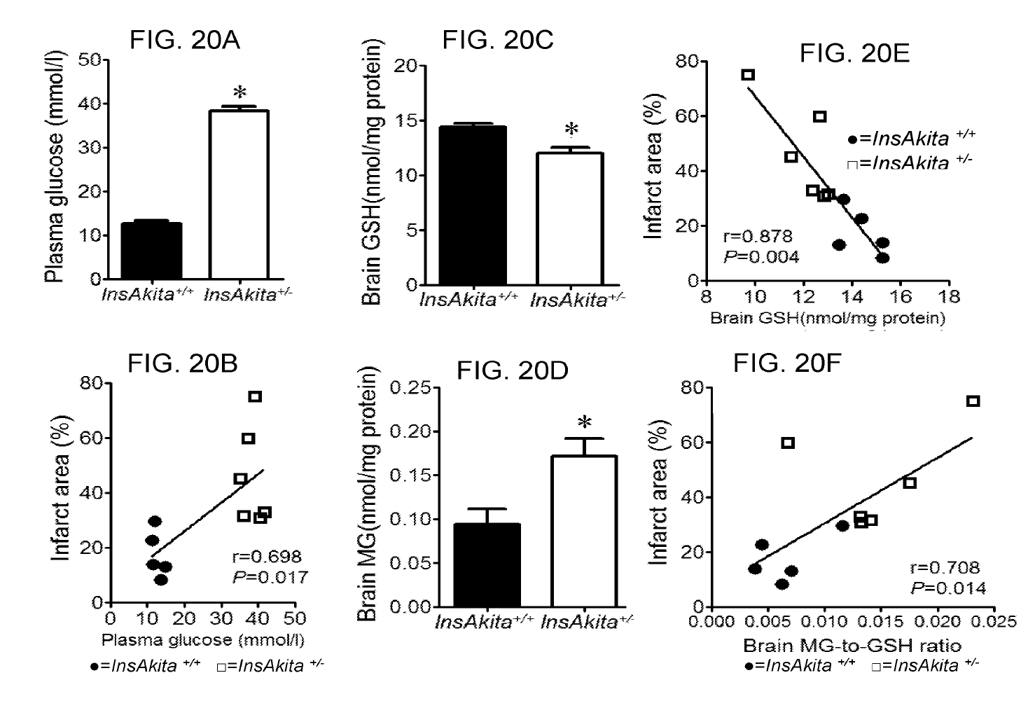

Turning to FIGS. 20A-20F, measurements in a genetic (InsAkita^{+/-}; n=6) model of diabetes compared to their respective controls (Veh; n=7, InsAkita^{+/+}; n=5) is shown. FIG. 20A shows plasma glucose levels. FIG. 20B shows correlations of percent brain infarct area with plasma glucose levels. FIG. 20C shows brain GSH levels. FIG. 20D shows brain MG concentrations. FIG. 20E shows correlations of infarct area with GSH. FIG. 20F shows correlations of infarct area with MG-to-GSH ratio. *P<0.05 InsAkita^{+/-} vs. InsAkita^{+/+}. Correlations: black circles=non-diabetic; white squares=diabetic.

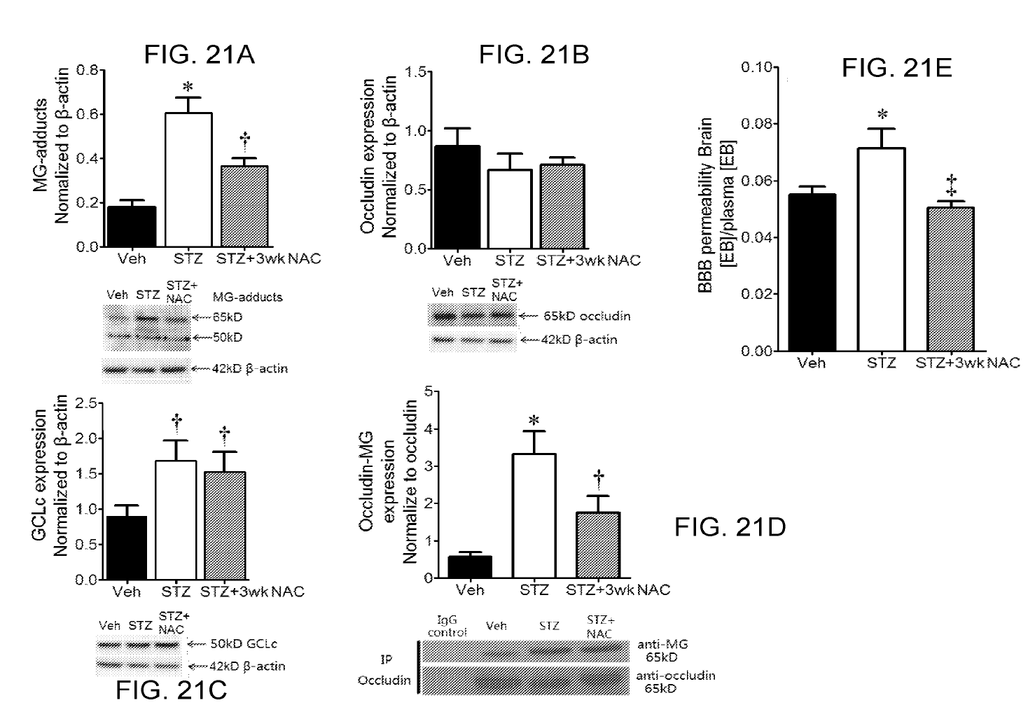

Turning to FIGS. 21A-21E, expression of MG-protein adducts (FIG. 21A), occludin (FIG. 21B) and GCLc (FIG. 21C) in the brain of non-diabetic (Veh) and diabetic (STZ) mice, as measured by Western blot is shown. Representative immunoblots are shown. The bar graphs show the quantitation of the protein band intensities normalized to β-actin; n=5/grp. FIG. 21D shows immunoprecipitation for occludin followed by immunoblot for MG, with quantification of the MG band intensity normalized to occludin. FIG. 21E shows the blood-brain barrier (BBB) permeability measured by Evans Blue extrusion in control and diabetic mice−/+NAC, n=4/grp. *P<0.05 vs. Veh and STZ+3 wk NAC; †P<0.05 vs. Veh; ‡P<0.05 vs. STZ.

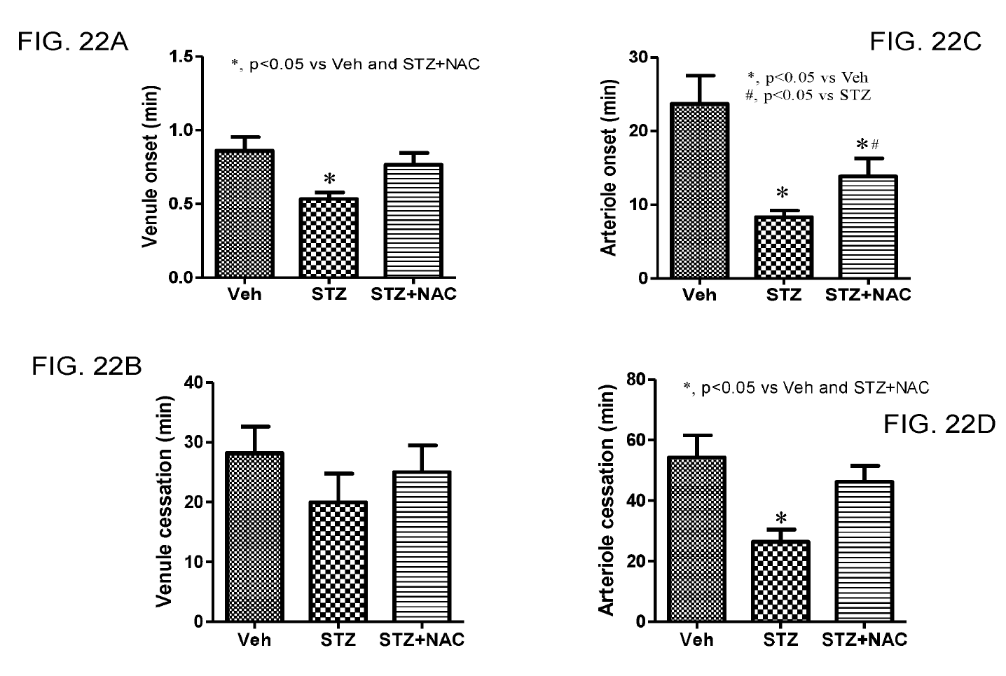

Turning to FIGS. 22A-22D, the thrombosis onset and cessation times in cerebral venules (FIGS. 22A and 22B) and arterioles (FIGS. 22C and 22D) of vehicle-treated non-diabetic mice (Veh), untreated diabetic mice at 6 weeks diabetes (STZ) or STZ-6 week mice treated with NAC (2 mM in the drinking water for 3 weeks) (STZ+NAC) is shown.

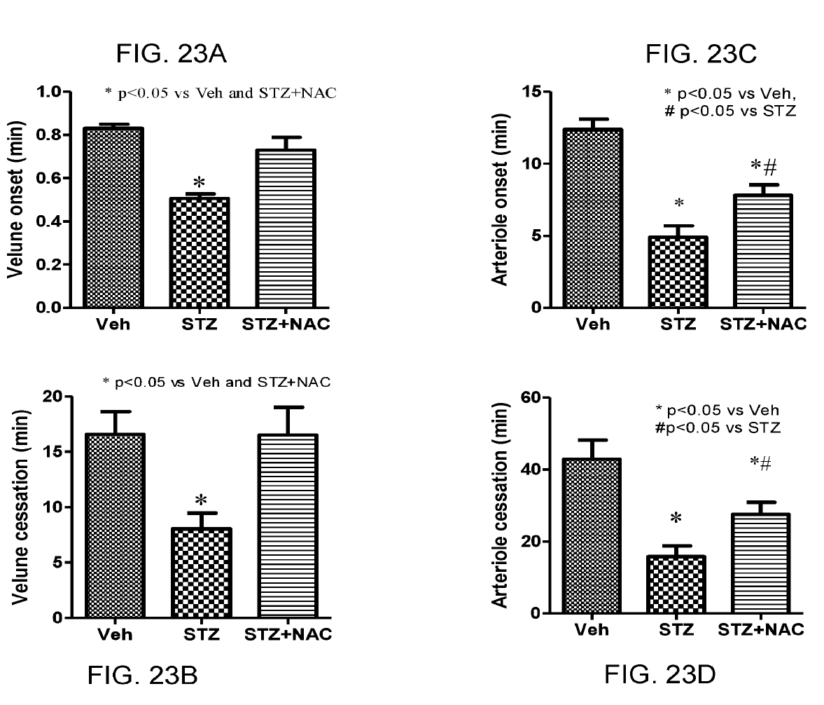

Turning to FIGS. 23A-23D, the thrombosis onset and cessation times in cerebral venules (FIGS. 23A and 23B) and arterioles (FIGS. 23C and 23D) of vehicle-treated non-diabetic mice (Veh), untreated diabetic mice at 20 weeks diabetes (STZ) or STZ-20 wk mice treated with NAC (2 mM in the drinking water for 3 weeks) (STZ+NAC) is shown.

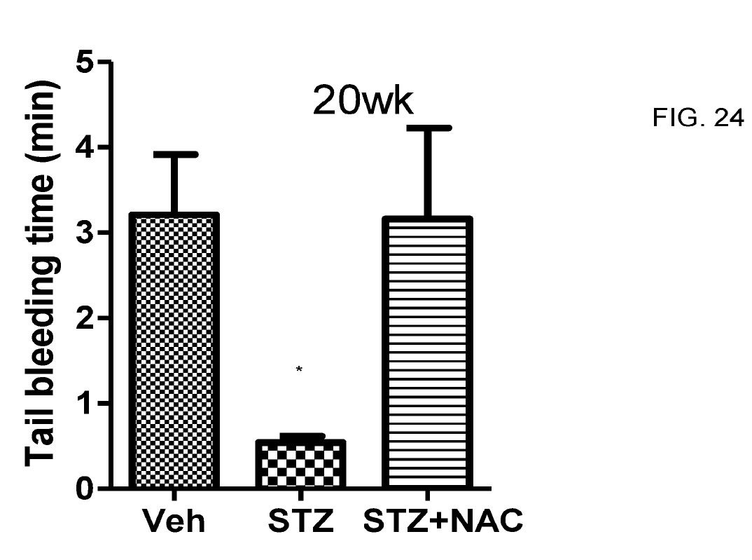

Turning to FIG. 24, as a measure of coagulation, tail bleed time was determined in animals that were diabetic for 20 weeks. An acceleration of tail bleed time was observed in the more chronic diabetic mice, and this was reversed by treatment with NAC (2 mM in the drinking water for 3 weeks). *P<0.05 vs. Veh and STZ+NAC.

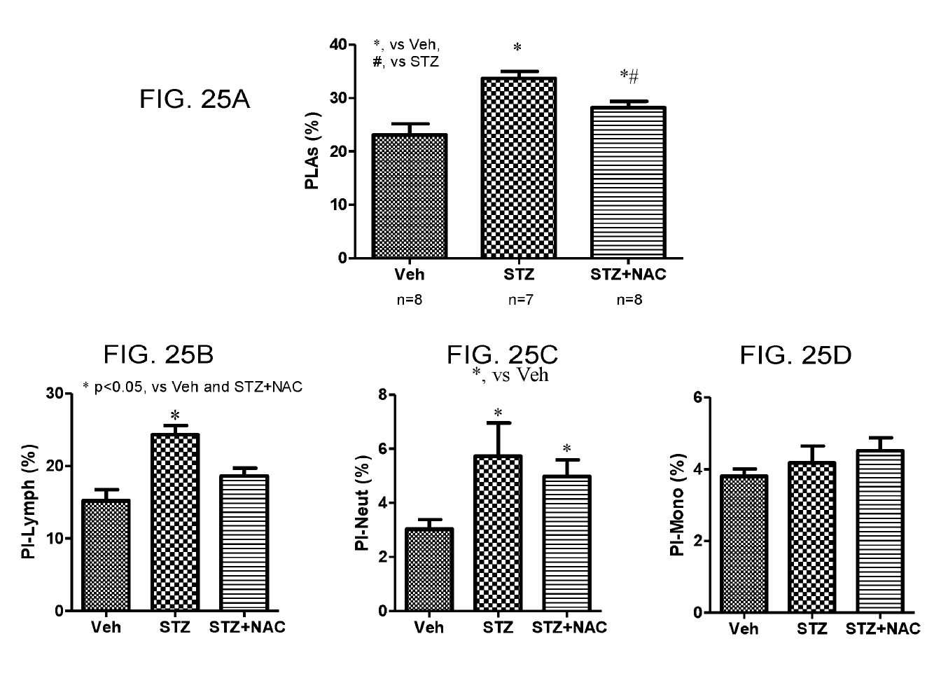

Turning to FIGS. 25A-25D, the percent of circulating leukocytes forming aggregates with platelets at 5 weeks of diabetes is shown (FIG. 25A). Aggregate formation between platelets and leukocyte subpopulations include: lymphocytes (FIG. 25B), neutrophils (FIG. 25C) and monocytes (FIG. 25D). Non-diabetic (vehicle-treated (Veh)), diabetic (STZ) and diabetic mice treated with NAC (2 mM for 2 weeks) were assessed.

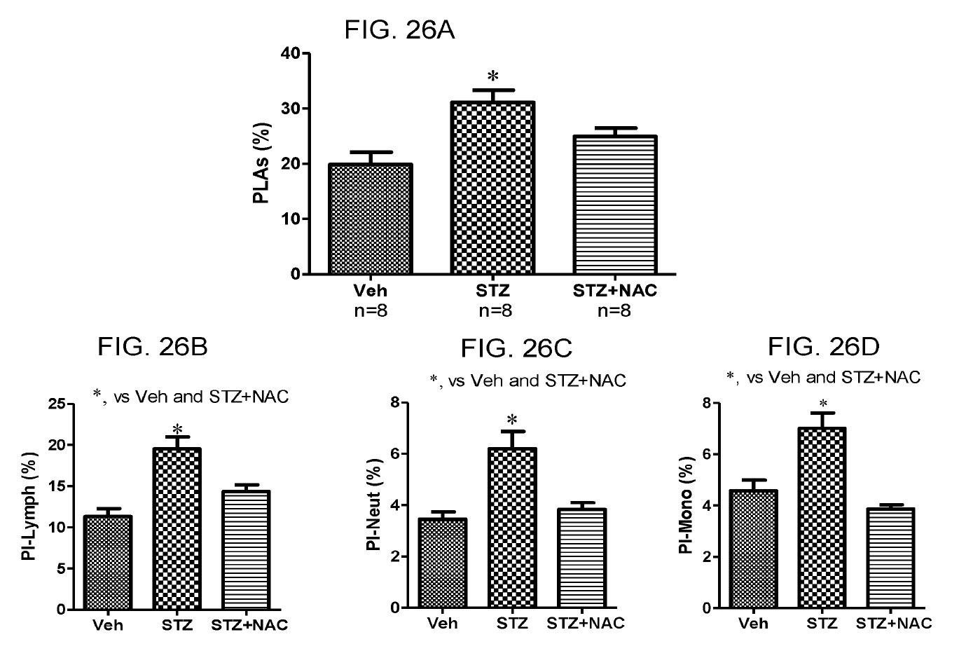

Turning to FIGS. 26A-26D, the percent of circulating leukocytes forming aggregates with platelets at 19 weeks of diabetes is shown (FIG. 26A). Aggregate formation between platelets and leukocyte subpopulations include: lymphocytes (FIG. 26B), neutrophils (FIG. 26C) and monocytes (FIG. 26D). Non-diabetic (vehicle-treated (Veh)), diabetic (STZ) and diabetic mice treated with NAC (2 mM for 2 weeks) were assessed.

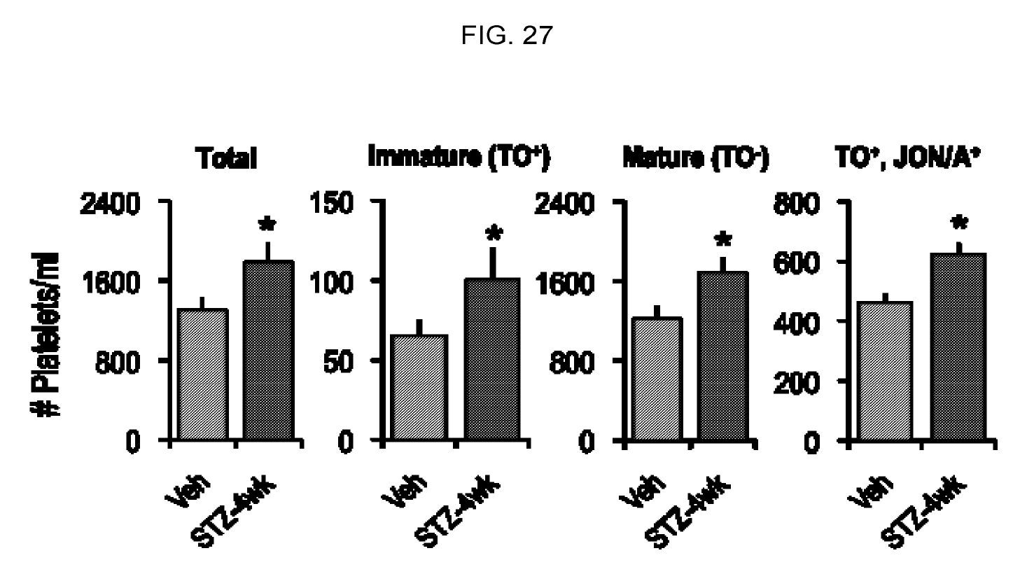

As shown in FIGS. 27-29, further aspects of platelet action, and NAC function alone and synergistic effects when combined with established diabetes treatments, were shown.

Based on the inventor's findings in the experimental mouse model of diabetes, discussed further below, the inventors disclose a novel use of the orphan drug, NAC in at least diabetic individuals for at least the purposes of preventing carbonyl stress (i.e., formation of protein cross-links with reactive carbonyl species) and cerebrovascular disease pathology, notably stroke. The inventors anticipate that this novel, preferably oral, intervention could represent a critical first step in the successful management of cerebrovascular risk and stroke outcome in diabetes.

Diabetes is a clinically important risk factor for cardiovascular disease (CVD) and cerebrovascular diseases (such as stroke), and is a leading and growing global health concern. About 382 million (8.3%) of the adult population worldwide has diabetes, a statistic that is anticipated to double in the next 25 years. North America has the highest comparative prevalence rates, at 9.3%. In 2012, 21 million adults (11%) in the United States were diagnosed with diabetes, one of the leading causes of death. Another 8 million people are estimated to be undiagnosed. The total economic cost of diagnosed cases alone totaled over $245 billion, a 41% increase over estimates in 2007. Significantly, new diabetic cases (with an alarming rate in children) and associated medical costs for disease management continue to rise annually.

Several limitations (e.g., inadequate glycemic control and side effects including hypoglycemia and gastrointestinal problems) hamper the efficacy of current treatments. Many of the treatments target symptoms rather than underlying mechanisms, an issue that is highlighted by the interesting and unresolved fact that even diabetic patients with well-controlled blood sugar levels exhibit an increased CVD risk. Of relevance to the current disclosure, the inventors' findings support a paradigm that diabetes is associated with enhanced stroke risk (e.g., increased thrombosis) and poor stroke outcome (e.g., increased brain infarct area). Diabetics also experience increased risk for other events that are secondary to the loss of platelet homeostasis and enhanced thrombosis. These include microvascular complications such as nephropathy, retinopathy, ischemia of the extremities e.g. mesenteric ischemia, atrial fibrillation (which can lead to emboli), and macrovascular disease such as CVD, and peripheral artery disease.

The diabetic condition is characterized by hyperglycemia and elevated plasma levels of reactive carbonyl species. Among the major reactive dicarbonyl species is MG, a potent glycating (cross-linking) agent, capable of inducing significant carbonyl stress. Indeed, MG is a precursor of advanced glycation end products (AGEs), notably HbAlc. MG is formed from triosephosphates from the metabolism of glucose; therefore a hyperglycemic status in diabetes would contribute to elevated MG levels and consequently, enhanced protein carbonyls (protein-glycated adducts). Thus, an increase in protein carbonyls (marker of carbonyl stress) in diabetes is implicated as a primary etiologic factor in the pathogenesis of diabetic microvascular diseases and associated complications. Under normal conditions, cellular removal of MG is highly efficient, in a process that relies on the availability of GSH, an essential intracellular cofactor in the glyoxalase pathway. However, tissue GSH was found to be notably decreased in diabetes.

N-acetylcysteine (NAC, structure given in FIG. 11) is a precursor of cysteine in cellular production of GSH (a tripeptide molecule consisting of glutatmate-cysteine-glycine). The inventors' experiments evidence that orally administered NAC (in drinking water) to 4- or 20 wk diabetic mice significantly (a) protects the diabetic brain against injury induced by I/R and (b) decreases the accelerated onset of thrombosis in brain microvessels, respectively (FIGS. 12A-13B). It is well known in the art that such mouse studies are strong evidence of efficacy of treatment in humans. Mechanistically, it was found that NAC increases brain GSH contents (FIG. 14A), decreases the accumulation of free MG (FIG. 14B), and attenuates the formation of protein carbonyls (FIG. 14C) in the diabetic brain. Significantly, post I/R brain injury (% infarct area) is directly correlated with the potential for MG detoxication by GSH (expressed as MG-to-GSH ratio, FIG. 15). That is, the lower the MG-to-GSH ratio, the lower the post I/R injury.

Unlike other post-translational modifications of proteins (e.g., phosphorylation, glutathionylation or nitrosylation), protein glycation appears to be an irreversible and deleterious event that results in a permanent alteration in protein function. In western blot analyses of diabetic brain extracts (FIG. 8A), the inventors discovered two major MG-glycated proteins that corresponded to the molecular sizes of (a) occludin, a pivotal member of the tight junctional complex of brain microvascular endothelium, and (b) the catalytic subunit of GCL, the rate-determining enzyme in the synthesis of GSH. Moreover, immunohistochemical staining of glycated proteins (i.e., MG-protein adducts) in cerebral microvessels in vivo in brains from vehicle-treated and diabetic mice at 8 weeks post-STZ treatment indicated a significantly higher number of MG-positive microvessels in the diabetic brain (FIGS. 16A-16E). This result is consistent with an enhanced vulnerability of the brain microvasculature to glycation (i.e., carbonyl stress) in diabetes. Take together, these findings imply that there exists a loss of the integrity of the microvascular endothelium of the blood-brain barrier and, consistent with decreased brain GSH in diabetes, a compromised capacity in the production of GSH in the diabetic brain.

The experimentally effective NAC dose was comparable to clinical dosage of NAC for various human diseases: The administered dose of 2 mM NAC in the inventors' mouse studies averaged approximately 0.2 mg/day/g mouse, based on volume of water intake and NAC concentration. This experimental NAC treatment regimen approximates clinical/therapeutic dosage of NAC for various human diseases, ranging from a lower value of 40 mg/day/kg (mg per day per kg mass of the human) to a mid-value of 80 mg/day/kg, and a high value of 160 mg/day/kg. A dose of 60 mg/kg/d was well-tolerated over the course of 8-75 months in young children. It is noteworthy that basal metabolism in mice is significantly higher than that of humans, and the inventors' experimental NAC dosage was within the same order of magnitude as current clinical/therapeutic levels in humans. Nevertheless, it is likely that a minimum or threshold dose of NAC that elicits beneficial effects in the human diabetic brain may be less than the lower value of 40 mg/day/kg. An important point is that MG can cause insulin insensitivity/resistance in many cell types, including endothelial cells, and insulin resistance is associated with worse inflammation and stroke severity in stroke patients. The use of NAC would decrease MG levels, and consequently, this would lower the required dose of insulin, or anti-glycemic drugs. As treatment continues and MG levels fall, NAC doses could be reduced, such that the maintenance doses of each drug (insulin or anti-glycemics) could be lower than what is currently used. The intake of lower drug doses would attenuate any drug-associated side effects and improve patient compliance.

This disclosure contains innovation in both concept and application. Current clinical use of NAC is focused on its mucolytic and antioxidant functions in the lung and liver, respectively. There is currently no known NAC treatment in the clinic for stroke, and no data for NAC usage in diabetes to decrease stroke risk or deleterious stroke outcome. The inventors disclose a new mechanism for NAC action in the brain in attenuating cerebrovascular pathophysiology. Mechanistically, the inventors find that NAC serves as a critical cysteine source to increase brain GSH, an essential co-factor in the elimination of reactive carbonyl species (such as MG) and in the prevention of protein-adduct formation.

The induction of carbonyl stress is a deleterious to the diabetic brain. An important protein target for MG-glycation could be the family of histones (FIG. 18). In previous cell studies, epigenetic marks on histones of the transcription factor NF-kB was implicated in diabetic glucose memory, an intriguing phenomenon resulting from a prior exposure to high plasma glucose. Histone turnover is slow, with an estimated half-life in the mouse brain of 223 days, suggesting that in the intact organism the effect of glucose could last for more than a year. It is anticipated that this timeline is longer in humans. NF-kB is likely a prime target of epigenetic changes in the diabetic brain, given the inventors' finding that adhesion molecules regulated by NF-kB are increased in diabetes (FIG. 17). A sustained pathological activation of NFkB would lead to downstream inflammatory pathways being turned on such as elevated release of cytokines that could enhance the susceptibility of the brain to worse outcome following stroke. Furthermore, there is a strong interaction between inflammation and platelets which could beget thrombosis, and increase the risk of stroke. Protein glycation is an irreversible process, and the key targets of glycation on proteins are the amino acids, lysine and arginine. Significantly, lysine and arginine comprise 15% of histone rendering the protein vulnerable to MG-glycation. The inventors propose that diabetic brain may be compromised by elevated MG-histone glycation and NF-kB activation, and that through GSH-dependent elimination of MG, NAC could eventually attenuate histone modifications and erase diabetes-associated glucose memory. Therefore NAC would confer a greater long term benefit in diabetes. In addition, other diseases, such as cancer that is associated with decreased GSH, high glucose utilization, and adverse epigenetic changes, could also benefit from NAC intervention.

Based at least on this new discovery, the inventors disclose a new use for NAC in attenuating stroke risk and deleterious stroke outcome. This new use for NAC will benefit at least three distinct populations of diabetic individuals, including diabetic individuals with poorly controlled glycemic status; non-diabetic individuals at high risk of developing diabetes; and diabetic individuals with well controlled glycemic status. These three groups represent a significant portion of the United States population.

First are diabetic individuals with poorly controlled glycemic status. The inventors' experimental data provides direct evidence that supports efficacy of orally administered NAC for this group of diabetic patients. The results in FIGS. 2A to 3B show that NAC affords significant protection in 4 week and 20 week diabetic mice against brain infarction after I/R and accelerated onset of thrombosis in brain microvessels, respectively. These results provide evidence that, despite an active disease state, diabetic patients given NAC would experience a lower stroke risk and those who suffer a stroke would have a better outcome than their diabetic counterparts not on NAC.

Second are individuals at high risk of developing diabetes. Based on the mechanism discovered, the experimental evidence strongly suggests that prophylactic intake of NAC by individuals at high risk for diabetes would, among other things, decrease the MG-to-GSH ratios in the brain, and thus lead to a lower stroke incidence and less severe outcome after a stroke incident in such individuals.

Third are diabetic individuals with well controlled glycemic status. It is an unresolved fact in this art that increased CVD risk remains in diabetic patients despite well-controlled blood sugar levels. This intriguing phenomenon has been attributed to glucose memory, which is believed to be controlled by epigenetic mechanisms wherein post-translational acetylation or methylation of transcription factors (such as NFKB) can result in inflammatory gene silencing or expression. Indeed, the inventors show that NFkB-dependent expressions of cellular adhesion molecules (like ICAM-1, E-selectin, and VCAM-1, FIGS. 17A-17D) were elevated in the diabetic brain, consistent with a proinflammatory phenotype in the diabetic brain microvasculature. Thus, increased carbonyl stress (evidenced by increased transcription factor histone lysine-MG adduction) would potentially “hijack” the normal epigenetic regulation. The inventors' studies in IHECs suggest that histone 3 may, in fact, be a susceptible target for glycation (FIG. 18), an irreversible event that will impact NF-kB-dependent inflammatory gene expression. In short, based on the current understanding in the art and the inventors' experiments, by promoting MG elimination through increased GSH, orally administered NAC would potentially “erase” glucose memory in this patient group with an anticipated reduced stroke risk and improved stroke outcome.

### Experiments

Materials

The following reagents were purchased from Sigma Chemical Company: glutathione, methylglyoxal, N-acetyl-cysteine, L-buthionine-(S, R)-sulfoximine, D-lactate, D-lactic dehydrogenase, glutamic-pyruvate transaminase, β-nicotinamide adenine dinucleotide hydrate, S-D-lactoylglutathione. The rabbit-anti-mouse GCLc antibody, rabbit-anti-mouse occludin antibody and HRP-conjugated goat-anti-rabbit IgG and anti-mouse MG antibody were purchased from Abcam, Invitrogen and JaICA, respectively. Anti-actin mouse antibody was obtained from BD Biosciences, HRP-conjugated sheep-anti-mouse and goat-anti-rabbit IgG from Amersham and Abcam, respectively. ECL reagent was purchased from BIO-RAD.

Animal Preparation

Four-week-old male C57BL/6J mice, weighing 18-20 g, were purchased from Jackson Laboratory. These mice were maintained at the Animal Facility of the Louisiana State University Health Sciences Center—Shreveport. Animal care and use in the experiments follow the guidelines in the Guide for the Care and Use of Laboratory Animals, published by the US National Institutes of Health. Following adaptation for 1 week, the mice were divided randomly into 4 groups: non-treated sham operation group (NT-sham group, n=6), non-treated I/R group (NT-I/R group, n=5), vehicle I/R group (Veh-I/R group, n=24), and streptozotocin I/R group (STZ-I/R group, n=19). Experimental diabetes was achieved in STZ-I/R group of mice by injecting 50 mg/kg of STZ for consecutive 5 days. On day 7, mice whose plasma glucose was more than 300 mg/dl were deemed to be diabetic. From diabetes onset at week 1, a group of diabetic mice were given 0.2 mM NAC in the drinking water for 3 weeks (n=3; a total of 4 weeks diabetes, 3 weeks NAC) or 1 week (n=9; a total of 2 weeks diabetes, 1 week NAC). Veh-I/R mice were injected with citrate buffer for consecutive 5 days, and a group of Veh-treated mice were given 0.2 mM NAC for 2 weeks in the drinking water before the ischemia operation (Veh+NAC group, n=8). Another Veh-treated mice were injected (intraperitoneally, 12 hr interval) with 0.5M BSO 24 h before the ischemia operation and during the 24 h reperfusion period (Veh+BSO group, n=10). All mice were 9 weeks old at sacrifice.

The Akita mice and GCLm mice were respectively gifts from Professor Harris and Pattillo at LSUHSC—Shreveport.

The Procedure of Middle Cerebral Artery Occlusion and Infarct Area Assessment

The left common, internal, and external carotid arteries were exposed, and the external carotid artery was ligated. A 6-0 silicone coated nylon filament was introduced through the common carotid artery. The filament was advanced into the internal carotid artery 9.0 to 11.0 mm beyond the carotid bifurcation until an elastic resistance indicated that the tip was properly lodged in the anterior cerebral artery and thus blocked blood flow to the MCA for 45 min (45 min ischemia). Then the filament was pulled out and the blood flow was restored for 24 h (24 h reperfusion). In sham operation, the filament was pulled out immediately once it was properly placed in the anterior cerebral artery.

The mice were euthanized by ketamine/xylasine at 24 h after reperfusion, and brains were quickly removed. The whole brain was cut into six 2-mm-thick slices evenly at sagittal view. The 1^{st}, 2^{nd}, 5^{th} and 6^{th} slices were rapidly frozen in liquid nitrogen for later analyses: HPLC, enzyme activity assay and western blot. The 3^{rd} and 4^{th} slices were incubated in 2% 2,3,5-triphenyltetrazolium chloride (TTC) solution for 30 minutes at 37° C., and pictures were obtained. The infarct area calculation was performed using the following formula: 100%×(contralateral hemisphere area-noninfarct ipsilateral hemisphere area)/contralateral hemisphere area. The final infarct area was expressed by the average of infarct area in the 3^{rd} and 4^{th} slices.

Quantification of GSH, Cysteine and γ-glutamylcysteine (γGC)

Tissue contents of GSH, cysteine and γGC were determined by high-performance liquid chromatography (HPLC). Briefly, brain tissues were harvested and homogenized in PBS and incubated with trichloroacetic acid (final concentration 10%) in 1.5-ml microcentrifuge tubes overnight at 4° C. The homogenates were centrifuged at 10000 rpm at 4° C. for 8 min. Then the supernatants were derivatized with 6 mM iodoacetic acid and 1% 2,4-dinitrophenyl fluorobenzene (adjusted pH to 7-8 and 7.0, respectively) to yield the S-carboxymethyl and 2,4-dinitrophenyl derivatives, respectively. Separation of GSH, cysteine and γGC derivatives whose peaks were detected at 365 nm wavelength was performed on a 250×4.6-mm Alltech Lichrosorb NH2 10 μm anion-exchange column. GSH, cysteine and γGC contents were quantified by comparison to standards derivatized in the same manner. Protein pellets were dissolved in 1 M NaOH for protein quantitation and GSH, cysteine and γGC concentrations were expressed as nanomoles per mg protein.

Quantification of MG

Tissue contents of MG were also determined by HPLC. The brain tissues were homogenized in the same manner as that for GSH measurement. Homogenates (581 μl) were incubated with 19 μl of 60% perchloric acid at room temperature for 24 h and then centrifuged at 12000 rpm at 4° C. for 10 min. The supernatants were derivatized with 0.1M of o-phenylenediamine for 24 h to produce 2-methylquinoxaline. On the third day, the supernatants were used for measurements of MG contents. Separation of MG derivatives, whose peak was detected at 315 nm wavelength, was performed on a 250×4.6-mm Chromegabond Ultra C-18 reversed phase HPLC column. The contents were quantified using MG standards derivatized with o-phenylenediamine. MG contents were normalized to protein concentrations and expressed as nanomoles per mg protein.

Assay of GCL Activity

Brain tissues were homogenized with TES/SE buffer containing 20 mM Tris, 1 mM EDTA, 250 mM sucrose, 20 mM sodium borate and 2 mM serine. The homogenates were centrifuged at 10000 g, at 4° C. for 10 min and the supernatants were transferred and centrifuged again at 15000 g, at 4° C. for 20 min. The final supernatants were used for assay of GCL activity. In brief, 80 μl of GCL reaction cocktail containing 400 mM Tris, 40 mM ATP, 20 mM L-glutamate, 2 mM EDTA, 20 mM sodium borate, 2 mM serine and 40 mM MgCl₂ was added to eppendorf tubes and pre-warmed to 37° C. (2 tubes for every sample, one reaction tube and one baseline tube). To every tube were added 80 μl aliquots of supernatant and 80 μl of 20 mM DTT and then pre-incubated for 5 min. Following preincubation, 80 μl of 2 mM cysteine was added to reaction tubes and 80 μl of TES/SB buffer to baseline tubes; all tubes were then incubated on an agitator for 45 min at room temperature. The reactions were stopped with 35.5 μl of 50% TCA and left at 4° C. overnight. The next processing steps were the same as those for GSH quantification. GCL activity equals the γGC concentration in the reaction tube minus that in the baseline tube and normalized to protein concentrations. GCL activity is expressed as nanomoles per mg protein.

Assay of Glyoxalase I and II Activity

Brain tissue was homogenized in 1:20 (weight/volume) of 10 mM Tris-HCl pH 7.4 containing a cocktail of proteinase inhibitors. The homogenates were centrifuged at 12000 g for 20 min at 4° C., and the supernatants were used for assay of enzyme activities.

Glyoxalase I activity assay was determined as S-D-lactoylglutathione (SDL) formation spectrophotometrically in 1-ml quartz cuvettes. The reaction system (total volume 1 ml) contained 182 mM imidazole buffer pH 7.0, 14.6 mM magnesium sulfate, 5 mM MG, 1.5 mM GSH and 35 μl tissue supernatant. SDL formation was monitored at 240 nm at 37° C. for 3 min, and quantified using the extinction coefficient of 13.6 mM⁻¹ cm⁻¹. Glyoxalase I activity was expressed as μmol SDL formed per min per mg protein.

Glyoxalase II activity assay was determined by GSH regeneration spectrophotometrically in 1-ml cuvettes. The reaction system contained 0.85 ml of 100 mM Tris-HCl pH 7.4 containing 0.8 mM SDL, 0.2 mM DTNB and 150 μl tissue supernatant. GSH formation was monitored at 412 nm at 37° C. for 3 min, and quantified using the extinction coefficient of 3.37 mM⁻¹ cm⁻¹. Glyoxalase II activity was expressed as μmol GSH formed per min per mg protein.

Western Blot Analyses of Occludin, GCLc, Actin, and MG-protein Adducts

Brain tissues were homogenized with RIPA lysis buffer containing 50 mM Tris, pH 8.0, 150 mM sodium chloride, 0.5% sodium deoxycholate, 0.1% SDS and protease inhibitor cocktail. Total protein (20 μg) per sample was loaded on 10% SDS-polyacrylamide gels. Electrophoresis was performed at 150V for 1 h, and transfer onto PVDF membranes at 100 V for 1 h. The membranes were blocked in 5% nonfat milk in TBST buffer containing 20 mM Tris, 137 mM NaCl, 0.1% Tween20, pH 7.6 for 40 min at room temperature followed by overnight incubation with either rabbit-anti-mouse occludin polyclonal antibody (1:2000), rabbit-anti-mouse GCGc polyclonal antibody (1:2000) or mouse anti-MG monoclonal antibody (1:2000) at 4° C. on an agitator. The second day, the PVDF membranes were incubated with HRP-conjugated goat-anti-rabbit or sheep-anti-mouse secondary antibody (1:10000) for 2 h at room temperature. Protein expression was detected using enhanced chemiluminescence (BIO-RAD) according to the manufacturer's instructions. The membranes were stripped and reprobed with anti-mouse actin monoclonal antibody (1:5000) to verify equal protein loading.

Assay of Protein Carbonyls

The extent of protein glycation was determined by measuring total protein carbonyl contents. Briefly, the brain homogenate was incubated with 10 mM of 2, 4-dinitro-phenylhydrazine (DNPH) in 2M hydrochloride acid (1:2 volume) for 1 h followed by precipitation with trichloroacetic acid (final concentration 10%). Following centrifugation, the precipitant was washed 3 times with ethanol-ethyl acetate (1:1) to remove free DNPH. The pellets were redissolved in 0.6 ml of 6 M guanidine hydrochloride solution containing 20 mM potassium phosphate (pH=2.3 adjusted with trifluoroacetic acid) at 37° C., and the insoluble materials were removed by centrifugation for 3 min at 11000 g. The absorbance was monitored at 366 nm, and protein carbonyl contents quantified using the extinction coefficient of 22000 M⁻¹ cm⁻¹.

Statistical Analysis

All data are mean±SE. The significance of difference was assessed by Student t test (single comparisons) or by one-way ANOVA with Newman-Keuls post hoc tests (multiple comparisons). Differences were considered significant at P<0.05.

Results

Diabetes Potentiates Ischemia-Reperfusion Brain Injury

The results are shown in FIG. 1. The inventors used two kinds of diabetic model: STZ-induced (chemical) and genetic model (Akita mice: genetic ablation of insulin II gene, type 1 diabetic model). It was found that STZ-induced diabetic mice had more serious brain infarct area after 45 min ischemia and 24 h reperfusion (P<0.05) than age-matched controls. The infarct areas in STZ-mice were ˜60%, as compared to 25% and 20% in the NT and Veh mice, respectively. There was no difference in brain injury between NT and Veh groups. I/R brain injury in Akita-DM mice was 40% compared with Akita non-diabetic mice (18%, P<0.05). Taken together the results demonstrate that the diabetic state (regardless of induction by chemical or genetic approaches) was associated with enhanced stroke outcome.

Cerebral Infarct Area Correlates With Plasma Glucose Levels

As shown in FIG. 2, the plasma glucose levels were significantly elevated after diabetes onset at 4 wk (529.9±26.3 mg/dl vs 205.7±8.1 mg/dl, P<0.001). The brain injury induced by I/R was correlated with the plasma glucose levels, and the scatter plot showed a positive linear correlation (r=0.766, P=0.0014). This suggests that hyperglycemia is a significant contributor to enhanced stroke outcome.

Cerebral Infarct Area Correlates With Brain GSH Levels and MG-to-GSH Ratio

As shown in FIGS. 3 and 4, the diabetic brain exhibited significantly lower tissue GSH levels (11.91±0.37 vs 9.79±1.06 nmol/mg protein, p=0.0276), but higher MG levels (0.1280±0.0182 vs 0.2218±0.0317 nmol/mg protein, p=0.0123) as compared to that in Veh-treated mice. Significantly, the increase in infarct area in diabetic brain was associated with decreases in tissue GSH, with a negative linear correlation (r=0.767, p=0.0031). Interestingly, % infarct area was not correlated directly with the tissue levels of MG per se (data not shown). Rather, although there was greater scatter, % infarct area in diabetic brain positively correlated with the MG-to-GSH ratio (linear correlation of r=0.677, p=0.0255), which means that I/R brain injury corresponded to a decrease in the availability of GSH for MG elimination.

The inventors used GCLm mice (genetic knockout of the modulatory subunit of GCL) to test the effects of GSH on infarct area. The results in FIG. 3 show that GCLm^{-/-} mice exhibited significantly lower GSH levels and higher post-I/R brain infarct area than GCLm^{+/+} mice (i.e., containing normal wild type GCL modulatory subunit) (P<0.05).

NAC Attenuates I/R Induced Diabetic Brain Infarct That Correlated With Increases in Tissue GSH Contents

As shown in FIG. 5, the infarct area was significantly attenuated in STZ+NAC mice as compared with age-matched mice treated with STZ alone. NAC-induced attenuation in infarct area was dramatic regardless of the duration of treatment, either 1 or 3 weeks (P<0.05), both resulted in significant increases in tissue GSH levels. Collectively, the results demonstrate a significant linear correlation between brain GSH and infarct area (r=0.573, P=0.006).

NAC Serves as a Cysteine Source for GSH Synthesis

As shown in FIG. 6, determination of tissue GCL activity (measured by formation of γ-GC, the first product formed from GCL-catalyzed reaction between glutamate and cysteine) showed no difference between Veh- and STZ-treated mice (4 wks), suggesting that overall GCL function does not appear to be altered by diabetes despite lower GSH contents. However, while there was no difference in γ-GC contents between control and diabetic brain, γ-GC contents trended lower in the diabetic brain. Collectively, the results suggest that the observed decrease in brain GSH in diabetes is unlikely to be due simply to changes in the GCL enzyme per se.

Measurements of tissue cysteine show that there was a trend for cysteine to decrease in STZ-induced mice compared with control mice. Surprisingly, treatment with NAC resulted in a significant decrease in the steady state levels of cysteine in both control (vehicle) and diabetic brain (FIG. 6B). Given that NAC increases GSH (see FIG. 5), the decrease in the free cysteine pool would be consistent with its consumption in GSH formation. Indeed, inhibition of GCL function with BSO treatment resulted in higher free cysteine levels, consistent with decreased cysteine utilization for GSH production upon GCL inhibition. There was no change in brain glutamate levels (data not shown) under all conditions. Taken together, these results suggest that increased availability of cysteine (such as with NAC) could be an important determinant in promoting GSH synthesis in the diabetic brain.

NAC Increases the GSH Potential for MG Elimination

As shown in FIG. 7, the inventors found that 3 week NAC treatment did not alter plasma glucose levels at 4 wk diabetes (despite significant protection against I/R brain injury at this time), suggesting that NAC protection was not through simply lowering or normalizing plasma glucose. The finding that NAC treatment was associated with high brain MG levels (although there was a trend for decreased MG, but not significant) was consistent with elevated glucose, a source of MG via glucose metabolism. Nevertheless, when expressed as MG-to-GSH ratio, it was notable that NAC significantly decreased this ratio, suggesting that NAC enhances the potential for GSH-catalyzed MG elimination. Consistent with this suggestion, it was noted that the extent of decrease in MG level paralleled the extent of increase in GSH following 3 week NAC treatment (also see FIG. 5).

Diabetes Increases GCLc Expression and MG-Protein Glycation

As shown in FIG. 8, expression of the occludin protein tended to be lower in diabetic mice compared with vehicle-treated mice (P>0.05), which is in agreement with decreased immunohistochemical staining for occludin content in cerebral microvessels in the diabetic brain (FIG. 16). Interestingly, GCLc protein expression was enhanced in diabetic mice compared with vehicle controls (P<0.05). Significantly, protein-MG adduction was increased in diabetic brain (P<0.05). It is notable that the molecular sizes of two primary MG glycated protein adducts corresponded to those of occludin and GCLc. These results are consistent with enhanced carbonyl stress in diabetes and with occludin and GCLc being vulnerable targets for MG glycation. Studies in IHECs suggest that histone 3 could be another important target protein for glycation (FIG. 18).

Diabetes Does Not Affect the Activity of Glyoxalase System

As shown in FIG. 9, brain activities of Glo I and II were not different between diabetic mice and Veh controls (both P>0.05), which suggests that diabetes had no impact on the activities of the glyoxalase enzymes per se.

Assay of Total Protein Carbonyls

As shown in FIG. 10, total protein carbonyl contents in the diabetic brain were significantly enhanced at 2 and 4 weeks after diabetes onset as compared to control mice (both P<0.05), which confirms that diabetes is associated with elevated carbonyl stress. Total protein carbonyl contents were higher at 4 wk diabetes, indicating that carbonyl stress increases with diabetes duration. Significantly, NAC was highly effective in attenuating the levels of protein carbonyls at 1 week post-treatment in 2-wk diabetic mice. In IHECs, NAC specifically was found to prevent the formation of MG-occludin adducts (FIG. 19). This means that early prevention of the formation of glycated protein adducts maybe a major protective mechanism of NAC in diabetes.

Discussion

Diabetes is a risk factor for cardiovascular and cerebrovascular diseases which are characterized by endothelial dysfunction. Clinically, diabetic patients are at high risk for stroke and cerebral small vessel disease. Diabetes mellitus is associated with higher mortality, worse functional outcome, more severe disability after stroke and a higher frequency of recurrent stroke. However, before the inventors' experiments, the underlying mechanisms were poorly understood by those in this field.

The inventors' experiments examined the effects of diabetes and roles of NAC on ischemic stroke using mouse models of diabetes and established models of I/R brain injury. The inventors' results showed that diabetes exacerbates cerebral injury after I/R, regardless of chemical or genetic modes of diabetes induction. Post-I/R infarct areas in diabetic brains were increased by at least 1.3-1.5 fold over control mouse brain.

A significant characteristic feature of the diabetic condition is hyperglycemia. The elevated blood glucose significantly potentiated the outcome of ischemic stroke in diabetics; this scenario is even evident in nondiabetic patients with high blood glucose values. This means that the blood glucose status is a determinant of ischemic stroke outcome. The inventors' results also showed that blood glucose was positively correlated with the brain infarct area. The observation that an increased CVD risk remained even in diabetic individuals with well controlled blood sugar levels, suggests the involvement of other factors. Reactive carbonyl species like MG is one such candidate. Diabetes is characterized by elevated plasma levels of MG, a precursor of advanced glycation end products, and MG-induced cross-linking (glycation) of proteins could contribute to stroke risk and disease outcome. MG was formed from glucose through glycolysis, and the inventors' data revealed that there was elevated protein carbonyl content in STZ-induced diabetic mouse brain tissue. MG can induce the oxidative stress, microvascular hyperpermeability and leukocyte recruitment, increase the expression of endothelial cell adhesion molecules P-selectin, E-selectin, intercellular adhesion molecule-1, damage the endothelial dysfunction, which could aggravate ischemic brain injury during diabetes.

MG is metabolized to D-lactate by the glyoxalase I/II system using GSH as a cofactor. As such, high levels of GSH should lessen I/R brain injury, as confirmed by the GCLm mouse studies; significantly lower % infarct area and higher brain GSH levels were found in GCLm^{+/+} mice as compared to GCLm^{-/-} mice. The inventors' data also show that brain GSH levels were significantly lower in STZ-induced diabetic mice, and that brain GSH levels are negatively correlated with brain infarct area. Treatment with a cysteine precursor, NAC in the drinking water, resulted in significant increases in brain GSH levels and attenuation of post-I/R brain infarct area.

To clarify the mechanism of GSH decrease in the diabetic brain, the inventors examined the expression and activity of GCL in brain tissue. The results show that GCLc protein expression was significantly increased in the diabetic brain, a likely consequence of diabetic oxidative stress-induced upregulation of NF-E2-related factor 2/antioxidant response element pathway and expression of antioxidant genes, such as GCL. It is noteworthy that despite increased GCL protein expression, GCL activity did not change in the diabetic brain, which suggests a compromised enzyme function. The glycation of GCLc could contribute to reduced enzyme activity. Further analysis showed that there was a tendency for cysteine to decrease in the diabetic brain. Surprisingly, treatment with NAC resulted in significant decreases in the steady state levels of cysteine in both control (vehicle) and diabetic brain. This was interpreted to mean that cysteine was being utilized for enhanced GSH synthesis in the presence of NAC, a cysteine source for γ-GC formation and consequently GSH production. This interpretation was supported by the finding that inhibition of GCL function with BSO treatment results in higher cysteine levels, consistent with decreased cysteine utilization for GSH production upon GCL inhibition. This latter result confirmed that NAC was an important cysteine source for GSH synthesis. The finding that there is no change in brain glutamate levels (data not shown) under all conditions was further evidence that cysteine, and not glutamate was the limiting substrate that control GSH levels. Thus, it appears that in diabetes, it was the availability of cysteine (substrate) rather than the amount of GCL (enzyme content) that was the major determinant of GSH content in the brain. Hence, promoting cysteine supply through NAC administration is an effective mode to enhanced brain GSH production in diabetes.

Overexpression of glyoxalase I can prevent vascular intracellular glycation, endothelial dysfunction and reduce hyperglycemia-induced formation of advanced glycation end products and oxidative stress in diabetic rats. Even in non-diabetic mice, the knockdown of Glo1 can elevate oxidative stress and MG modification of glomerular proteins to diabetic levels, and can cause alterations in kidney morphology that are indistinguishable from those caused by diabetes. These data confirm an essential role for the glyoxalase system during diabetes. Interestingly, the inventors found little difference in glyoxalase I and II activities in brain tissues of STZ-induced diabetic mice and control mice. Based on the findings with NAC, the inventors conclude that the supply of the co-factor, GSH maybe the determining factor in controlling the overall function of the glyoxalase system and MG elimination.

The functional integrity of the blood brain barrier is important in protecting the brain tissue against systemic influences. In diabetes, an increase in total protein carbonyl contents and enhanced MG-glycation of occludin at endothelial tight junctions could compromise the blood-brain barrier function and contribute to ischemic brain injury. NAC, through inhibiting protein glycation, affords protection against carbonyl stress-induced brain microvascular dysfunction.

### Further Experiments

The Correlation Between GSH, MG and Stroke Injury Discovered in a Genetic Model of Diabetes

The inventors further characterized the genetic model of diabetes (Akita mice) to show that, similar to the inventors' findings in the chemical model (FIG. 2), the plasma glucose levels were significantly higher in the diabetic mice when compared to their littermate controls (FIG. 20A). Furthermore, the brain injury following stroke was significantly correlated with the glucose levels (FIG. 20B). This provides further support for a role for hyperglycemia in the exacerbation of stroke injury by diabetes.

Also in line with what was found in the chemical model of diabetes, genetically diabetic mice had lower levels of GSH (FIG. 20C), but higher levels of MG (FIG. 20D). GSH levels inversely correlated with stroke injury (FIG. 20E), suggesting a role for the decreased tissue GSH in post-stroke outcome. In support of the importance of GSH in the elimination of MG, the MG:GSH ratio showed a linear positive correlation with the extent of brain injury following stroke (FIG. 20F). These results provide further support for the inventors' initial data in the chemical model of type I diabetes, and show that the inventors' findings are not limited to a specific model of diabetes, but rather may apply to all diabetes models.

NAC Protects Against Diabetes Induced MG-Adduct Formation, But Not GCLc Expression

The inventors have shown that diabetes increases GCLc expression and MG-protein glycation (FIG. 8). Further, expression of occludin, a protein important in maintaining a tight blood-brain barrier, tended to be lower. The inventors further found that while NAC does not alter the total protein expression of either occludin (FIG. 21B) or GCLc (FIG. 21C), MG-adduct formation is significantly decreased by NAC (FIG. 21A). Furthermore, based on the inventors' finding that one of the MG-protein adducts was 65 kDa (the molecular weight of occludin), immunoprecipitation was performed to determine if this protein was occludin. The inventors' results confirmed that diabetes caused a significant increase in occludin glycation (FIG. 21D). A likely pathophysiological consequence of these changes would be increased blood-brain barrier (BBB) permeability. In agreement with this, FIG. 21E shows that plasma-to-tissue leakage of Evans Blue in diabetic brains was higher than in non-diabetic counterpart, indicating a substantial BBB breach during diabetes. Brain water content was also elevated (data not shown). After treating with NAC for 3 weeks, both the occludin glycation (FIG. 21D) as well as the leakage of Evans Blue (FIG. 21E) were significantly decreased versus their untreated counterparts. These findings evidence that NAC protects proteins from glycation and that this preserves the protein function. In this way, NAC protects against the damage caused to vascular integrity by diabetes.

NAC protects against accelerated thrombosis in diabetes. Initial data (FIG. 12) suggested that NAC might also diminish stroke risk by preventing the acceleration of the thrombotic process by diabetes. FIGS. 22A to 23D show that both the time for the initiation of thrombosis (onset time) and the time for the blood flow to be completely blocked by the thrombus (cessation time) were faster in the diabetic mice. This was more evident in arterioles at the earlier time point of diabetes (FIGS. 22C and 22D). At the later stage of diabetes, both postcapillary venules and arterioles were affected (FIGS. 23A-D). Significantly, NAC provided protection against the accelerated thrombosis in both types of vessels. The efficacy of NAC at the later time point has important implications for diabetic patients, in that it shows NAC treatment that is started well after diabetes is established (i.e. relevant for most of the current diabetic population) may reduce risk for stroke or other thrombotic complications.

NAC protects against accelerated coagulation in diabetes. Tail bleed time was used as an indicator of the coagulation process. In mice that were diabetic for 20 wks, (but not 6 weeks—data not shown), diabetes caused clotting to occur more rapidly (FIG. 24). However, NAC prevented this accelerated coagulation, restoring to normal the time for the blood flow to stop after clipping the tail. The fact that the impact of diabetes was seen at 20 weeks of diabetes, when a greater impact of diabetes was also seen in venule thrombosis (vs. 6 weeks) is of interest, because thrombosis in venules relies more heavily on the coagulation process than thrombosis in arterioles. Thus the reversal of the effect by NAC may at least in part explain how NAC protects against venular thrombosis at this stage of diabetes.

NAC protects against platelet-leukocyte aggregate formation. Diabetes led to an increased interaction between leukocytes and platelets (i.e. formation of platelet-leukocyte aggregates (PLAs)) (FIGS. 25A-26D). This is an indication of activation of both cell types, as well as communication between the two blood cell populations. Furthermore, as diabetes progressed from 5 weeks (FIGS. 25A-25D) to 19 weeks (FIG. 26A-26D) (the samples were taken one week before thrombosis), not only neutrophils and lymphocytes formed aggregates with platelets, but platelet-monocyte aggregates also began to appear. Significantly, despite this change in PLA characteristics, NAC effectively normalized the exacerbated PLA formation in diabetic mice, and by 20 weeks all leukocyte subpopulations were affected. This evidences that, in addition to protecting the blood vessels, NAC is acting in the systemic circulation to protect against pro-thrombus changes.

STZ model: in FIG. 27 platelet activation is seen at the early time point (no NAC data). This supports the PLA data in FIGS. 25A to 26D.

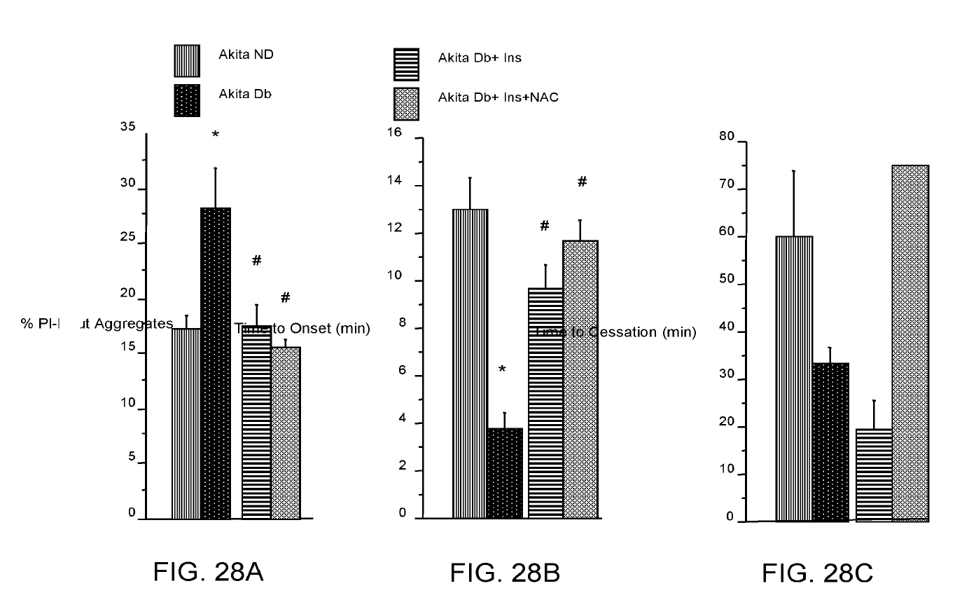

Akita model: like STZ model, shown in FIGS. 28A-28C percent platelet-neutrophil aggregate formation is increased, and time to onset and cessation in thrombosis are accelerated in arterioles and venules. Again this reiterates that the effects of diabetes may be “global” rather than confined to one type/cause of diabetes. Here the inventors used insulin treatment to control the glucose levels, and found this was associated with reversal of the pl-neut aggregate formation, and also the onset time in arterioles, but did not correct onset time in venules, or cessation time (time to complete occlusion of the vessel). However, when used with NAC, the showed complete protection against all parameters (no NAC only treatment for these mice). The data evidences that glucose control, which is the current treatment, is not completely effective and that co-treatment with NAC is very effective. In fact, NAC plus glucose control appears more effective than NAC alone in the STZ model in terms of how beneficial it is against thrombosis. It appears that insulin and NAC are additive/synergistic.

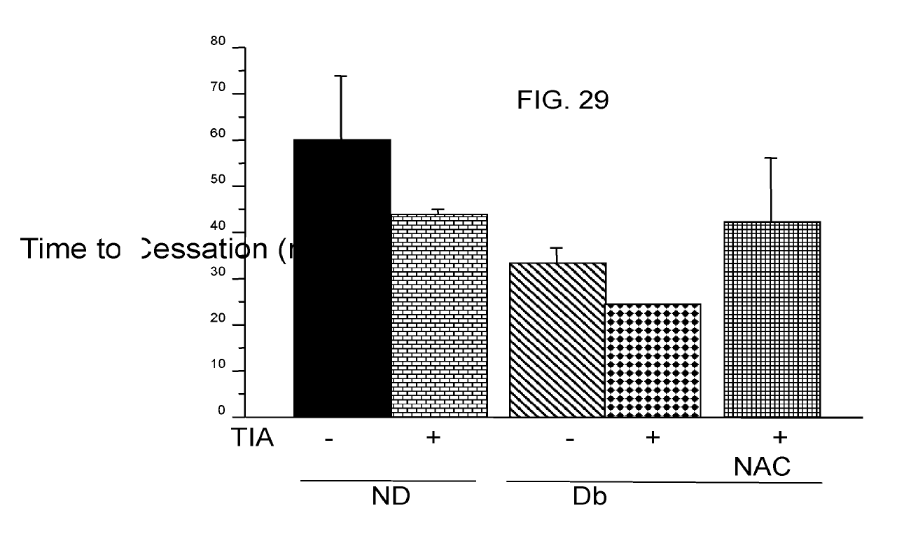

As shown in FIG. 29, the inventors also investigated the effect of transient ischemic attack (TIA, i.e. mini-strokes) on thrombosis, and found that in both non-diabetic and diabetic there was a tendency for slightly accelerated thrombosis versus no TIA. NAC showed some improvement of the thrombosis time in the diabetes+TIA (only group experimented with NAC). It is not yet apparent if the NAC protection it is acting on the diabetes effect alone, the TIA effect alone, or both the diabetes effect and the TIA affect. An importance of this lies in the fact that TIAs increase the vulnerability of a patient to a subsequent severe stroke. TIAs occur at a higher frequency in diabetic individuals, and render diabetics twice as likely to have a stroke within 90 days compared with non-diabetics. It is likely that the heightened platelet activation (shown by pl-leukocyte aggregates and activation above) and coagulation status (shown by tail bleed data) during diabetes lead to the accelerated thrombosis shown, and all three are attenuated by NAC. Since both TIA and stroke are thrombotic events, this has important therapeutic implications.

In conclusion, diabetes potentiates I/R brain injury. The mechanisms involve decreased brain GSH and increased MG levels. In diabetes, the increase in brain free MG levels was due to reduced GSH availability to support activity of the glyoxalase system, resulting in elevated MG-induced protein glycation and aggravated I/R organ injury. Diabetes-associated reduction in brain GSH is attributed to a decreased precursor cysteine pool. In addition, while diabetes increased the expression of GCLc, its function is likely to be compromised by MG glycation. Thus, decreased GSH substrate and reduced GCL function collectively contributed to lower GSH production in the diabetic brain. Orally administered NAC to diabetic animals, including humans, could provide the essential cysteine source for GSH synthesis, restore the potential for GSH-catalyzed MG elimination, and prevent the formation of glycated protein adducts, including GCLc.

While various embodiments of the present invention have been described in detail, it is apparent that various modifications and alterations of those embodiments will occur to and be readily apparent to those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/We claim:

1. A method for one of treating, preventing, and minimizing ischemia/reperfusion injury in a mammal, comprising:
administering to the mammal an effective amount of N-acetylcysteine (NAC).

2. The method of claim 1 further comprising the mammal having diabetes

3. The method of claim 1 further comprising the mammal being a human.

4. The method of claim 1 further comprising the effective amount being between one of 40 mg NAC per day per kg mass of the mammal to 80 mg NAC per day per kg mass of the mammal and 80 mg NAC per day per kg mass of the mammal to 160 mg NAC per day per kg mass of the mammal, given individually or combination with reduced doses of current diabetes therapies, including as insulin and anti-glycemic drugs.

5. The method of claim 1 further comprising a mode of administration being orally or intravenously.

6. The method of claim 1 further comprising administering before disease (prophylactic) in at-risk populations, during active disease/pathology and during disease resolution.

7. A method for one of preventing and minimizing diabetes pathology in a mammal, comprising:
administering to the mammal an effective amount of N-acetylcysteine (NAC).

8. The method of claim 7 wherein the diabetes pathology is one of associated microvascular disorders, including nephropathy, retinopathy and/or neuropathy, associated macrovascular disorders, including peripheral artery disease, cardiovascular disease, and associated glycemic control disorders due to diabetes-associated glucose memory and epigenetic changes.

9. The method of claim 7 further comprising the step of administering a chemical to the mammal to control blood glucose.

10. The method of claim 9 wherein the chemical is insulin.

11. The method of claim 9 wherein the chemical is one of a biguanide, a sulfonylurea, a meglitinide, a D-phenylalanine derivative, a thiazolidinedione, a DPP-4 inhibitor, an alpha-glucosidase inhibitor, a bile acid sequestrants, such as colesevelam, and a combination thereof.

12. A method for one of preventing, treating, and minimizing a disease conditions in a mammal, comprising:
administering to the mammal an effective amount of N-acetylcysteine (NAC);
wherein, the disease condition is associated with one of an elevated methylglyoxal level and a decreased tissue glutathione level.

13. The method of claim 12 wherein the disease condition includes one of cataracts, uremia, peritoneal dialysis and liver cirrhosis.

14. The method of claim 12 further comprising the step of administering to the mammal an effective amount of glutathione.

15. The method of claim 12 wherein the disease condition includes cancer, insulin resistance, metabolic diseases, obesity, thrombotic/thromboembolic pathologies, neurodegenerative disorders, dementia, Alzheimer's, Parkinson's, surgical ischemic episodes, transplant, and bypass.
